(19) United States
(12) Patent Application Publication
(10) Pub. No.: US 2026/0256781 A1
(43) Pub. Date: Sep. 3, 2026

(54) TREATMENT OF ANTIDEPRESSANT RESISTANT SUBJECTS

(71) Applicant: ACTINOGEN MEDICAL LIMITED, Sydney NSW (AU)

(72) Inventor: Steven GOURLAY, Sydney NSW (AU)

(21) Appl. No.: 18/872,904

(22) PCT Filed: Jun. 9, 2024
(86) PCT No.: PCT/AU2023/050513

§ 371 Date: Dec. 9, 2024

(30) Foreign Application Priority Data
Jun. 10, 2022 (AU) 2022901596

(51) Int. Cl. A61K 31/506; A61P 25/24; A61P 25/28

(52) U.S. Cl. CPC A61K 31/506; A61P 25/24; A61P 25/28

(57) ABSTRACT

The present disclosure generally relates to the surprising discovery that azabicyclothiophenyl compounds can treat depression in subjects resistant to antidepressant treatments and/or treat depression associated cognitive impairment in subjects resistant to antidepressant treatments.

### CROSS-REFERENCE TO RELATED APPLICATION

The present application claims priority from Australian Provisional Patent Application No. 2022901596 filed on 10 Jun. 2022, the contents of which are incorporated herein by reference in their entirety.

### FIELD

The present disclosure generally relates to the pharmaceutical field of treating depression in subjects resistant to antidepressant treatment and/or treating depression associated cognitive impairment in subjects resistant to antidepressant treatment.

### BACKGROUND

Major Depressive Disorder (MDD) involves the presence of major depressive episodes not being part of any psychotic or bipolar disorder. Symptoms of a major depressive episode include depressed mood; markedly diminished interest or pleasure in all, or almost all, activities most of the day; weight loss when not dieting or weight gain, or decrease or increase in appetite nearly every day; insomnia or hypersomnia nearly every day; psychomotor agitation or retardation nearly every day; fatigue or loss of energy nearly every day; feelings of worthlessness or excessive or inappropriate guilt nearly every day; diminished ability to think or concentrate, or indecisiveness, nearly every day; recurrent thoughts of death, recurrent suicidal ideation without a specific plan, or a suicide attempt or a specific plan for committing suicide. Further, the symptoms cause clinically significant distress or impairment in social, occupational, or other important areas of functioning (Diagnostic and Statistical Manual of Mental Disorders, 5th Edition, American Psychiatric Association, 2013).

Current treatment options for depression include monotherapy or combination therapy with various classes of drugs including mono-amine oxidase inhibitors (MAOI), tricyclic antidepressants (TCA), serotonin specific reuptake inhibitors (SSRI), serotonin noradrenergic reuptake inhibitors (SNRI), noradrenaline reuptake inhibitor (NRI), “natural products” (such as Kava-Kava, St. John's Wort), dietary supplement (such as s-adenosylmethionine) and others. More specifically, drugs used in the treatment of depression include, but are not limited to imipramine, amitriptyline, desipramine, nortriptyline, doxepin, protriptyline, trimipramine, maprotiline, amoxapine, trazodone, bupropion, chlomipramine, fluoxetine, citalopram, sertraline, paroxetine, tianeptine, nefazadone, venlafaxine, desvenlafaxine, duloxetine, reboxetine, mirtazapine, phenelzine, tranylcypromine, and/or moclobemide. Several of these agents including, but not limited to, serotonin reuptake inhibitors are also used when depression and anxiety co-exist, such as in anxious depression.

In the clinic, up to about 50% of depressed patients who are initially prescribed antidepressant therapy do not experience a timely remission of depression symptoms. This group typifies level 1 treatment-resistant depression, that is, a failure to demonstrate an “adequate” response to an “adequate” treatment trial. About 30% of depressed patients remain partially or totally treatment-resistant to at least two antidepressant treatments including combination treatments. Increasingly, treatment of treatment-resistant depression includes augmentation strategies including treatment with pharmacological agents such as, antipsychotics (such as quetiapine, aripiprazole, olanzapine, risperidone, and the like), lithium, carbamazepine, and triiodothyronine; adjunctive electroconvulsive therapy; adjunctive transcranial magnetic stimulation; etc.

Accordingly, there remains a need for alternative and effective treatments for treating depression in subjects resistant to antidepressant treatment including treating depression associated cognitive impairment.

### SUMMARY

The present disclosure is predicated in part on the surprising discovery that azabicyclothiophenyl compounds can treat depression in subjects resistant to antidepressant treatments and/or treat depression associated cognitive impairment in subjects resistant to antidepressant treatments.

Accordingly, in a first aspect, there is provided a method of treating or preventing depression in a subject resistant to antidepressant treatment, comprising administering to the subject a therapeutically effective amount of a compound of Formula I, or a pharmaceutically acceptable salt, solvate or prodrug thereof:

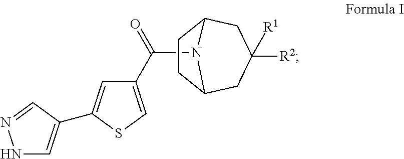

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are each independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

In a second aspect, there is provided a method of treating or preventing depression associated cognitive impairment in a subject resistant to antidepressant treatment, comprising administering to the subject a therapeutically effective amount of a compound of Formula I, or a pharmaceutically acceptable salt, solvate or prodrug thereof:

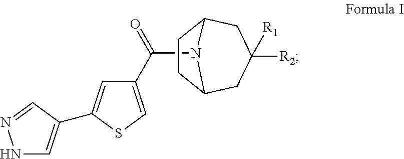

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are each independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

In one example, the treatment is for depression and depression associated cognitive impairment in the subject.

In one example, the compound of Formula Ia is:

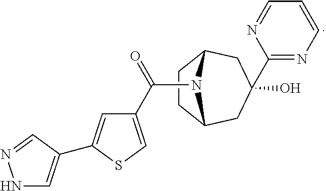 [SMILES: O=C(c1csc(-c2cn[nH]c2)c1)N1[C@@H]2CC[C@H]1C[C@](O)(c1ncccn1)C2]

In another aspect, there is provided use of a compound of Formula I or Formula Ia according to any examples or embodiments thereof as described herein for treating depression in subjects resistant to antidepressant treatments and/or treating depression associated cognitive impairment in subjects resistant to antidepressant treatments.

In another aspect, there is provided use of a compound of Formula I or Formula Ia according to any examples or embodiments thereof as described herein in the manufacture of a medicament for treating or preventing depression in subjects resistant to antidepressant treatments and/or treating depression associated cognitive impairment in subjects resistant to antidepressant treatments.

In another aspect, there is provided a compound of Formula I or Formula Ia according to any examples or embodiments thereof as described herein for use in treating or preventing depression in subjects resistant to antidepressant treatments and/or treating depression associated cognitive impairment in subjects resistant to antidepressant treatments.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### DETAILED DESCRIPTION

### General Definitions

Unless specifically defined otherwise, all technical and scientific terms used herein shall be taken to have the same meaning as commonly understood by one of ordinary skill in the art (e.g., chemistry, biochemistry, medicinal chemistry, microbiology and the like).

As used herein, the term “and/or”, e.g., “X and/or Y” shall be understood to mean either “X and Y” or “X or Y” and shall be taken to provide explicit support for both meanings or for either meaning, e.g. A and/or B includes the options i) A, ii) B or iii) A and B.

As used herein, the term about, unless stated to the contrary, refers to +/−20%, typically +/−10%, typically +/−5%, of the designated value.

As used herein, the terms “a”, “an” and “the” include both singular and plural aspects, unless the context clearly indicates otherwise.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will prevail. In addition, the materials, methods, and examples are illustrative only and not intended to be limiting.

As used herein, the term “treating” (or “treat”, “treatment” etc.) includes a reduction, alleviation and/or elimination of one or more symptoms associated with a specific disorder or condition. Such symptoms may be correlated with depression and depression associated cognitive decline or impairment in subjects showing at least some resistance to antidepressant treatments. For example, as used herein, the phrase “treating cognitive impairment” includes improving, reducing, alleviating and/or eliminating symptoms associated with cognitive impairment, relative to the symptoms prior to treatment.

As used herein, the term “preventing” (or “prevention”) includes prophylaxis of the specific disorder or condition. For example, as used herein, the phrase “preventing cognitive impairment” refers to preventing the onset or duration of the symptoms associated with cognitive impairment in subjects. In some embodiments, the phrase “preventing cognitive impairment” refers to slowing or halting the progression of cognitive impairment. In some embodiments, the phrase “preventing cognitive impairment” refers to delaying or preventing the onset of the symptoms of the cognitive impairment. Prevention may be absolute (such that no cognitive impairment occurs), or may be effective only in some individuals, to some extent, or for a limited amount of time.

As used herein, the term “subject” may be used interchangeably with the terms “patient” and “individual”. In some embodiments, the subject is a mammal. In some embodiments, the subject is a human.

As used herein, the term “halogen” means fluorine, chlorine, bromine, or iodine.

As used herein, the term “alkyl” encompasses both straight-chain (i.e., linear) and branched-chain hydrocarbon groups. Examples of alkyl groups include, but are not limited to, methyl, ethyl, n-propyl, iso-propyl, n-butyl, t-butyl, i-butyl, sec-butyl, pentyl, and hexyl groups. In one example, the alkyl group is of one to six carbon atoms (i.e. C₁₋₆alkyl).

As used herein, the term “alkoxy” refers to the group —O-alkyl, where “alkyl” is as described above. Examples of alkoxy groups include, but are not limited to, methoxy, ethoxy, propoxy, and butoxy groups. In one example, the alkoxy group is of one to six carbon atoms (i.e. —O—C₁₋₆alkyl).

As used herein, the term “alkenyl” refers to both straight and branched chain unsaturated hydrocarbon groups with at least one carbon-carbon double bond. Examples of alkenyl groups include, but are not limited to, ethenyl, propenyl, butenyl, pentenyl, and hexenyl groups. In one example, the alkenyl group is of two to six carbon atoms (i.e. C₂₋₆alkenyl).

As used herein, the term “alkynyl” refers to both straight and branched chain unsaturated hydrocarbon groups with at least one carbon-carbon triple bond. Examples of alkynyl groups include, but are not limited to, ethynyl, propynyl, butynyl, pentynyl, and hexynyl groups. In one example, the alkynyl group is of two to six carbon atoms (i.e. C₂₋₆alkynyl).

As used herein, the term “haloalkyl” refers to an alkyl group having at least one halogen substituent, where “alkyl” and “halogen” are as described above. Similarly, the term “dihaloalkyl” means an alkyl group having two halogen substituents, and the term “trihaloalkyl” means an alkyl group having three halogen substituents. Examples of haloalkyl groups include fluoromethyl, chloromethyl, bromomethyl, iodomethyl, fluoropropyl, and fluorobutyl groups. Examples of dihaloalkyl groups include difluoromethyl and difluoroethyl groups. Examples of trihaloalkyl groups include trifluoromethyl and trifluoroethyl groups. In one example, the haloalkyl group is of one to six carbon atoms (i.e. C₁₋₆haloalkyl).

As used herein, the term “oxyhaloalkyl” refers to the group —O-haloalkyl, where “haloalkyl” is as described above. Examples of —O-haloalkoxy groups include —O-fluoromethyl, —O-chloromethyl, —O-bromomethyl, —O-iodomethyl, —O-fluoropropyl, and -Ofluorobutyl groups. In one example, the oxyhaloalkyl group is of one to six carbon atoms (i.e. —O—C₁₋₆haloalkyl).

As used herein, the term “carbocyclyl” refers to an aromatic or non-aromatic cyclic group of carbon atoms. A carbocyclyl group may, for example, be monocyclic or polycyclic (i.e. bicyclic, tricyclic). A polycyclic carbocyclyl group may contain fused rings. In one example, the carbocyclyl group is of three to ten carbon atoms (i.e. C₃-iocarbocyclyl). In one example, the carbocyclyl group is of three to seven carbon atoms (i.e. C₃₋₇carbocyclyl). Examples of monocyclic non-aromatic carbocyclyl groups include cyclopropyl, cyclobutyl, cyclopentyl, cyclopentenyl, cyclohexyl, cyclohexenyl, cycloheptyl, and cyclooctyl groups. Aromatic carbocyclyl groups include phenyl and napthalenyl.

As used herein, the term “heterocyclyl” refers to an aromatic or non-aromatic cyclic group which is analogous to a carbocyclic group, but in which from one to three of the carbon atoms is/are replaced by one or more heteroatoms independently selected from nitrogen, oxygen, or sulfur. A heterocyclyl group may, for example, be monocyclic or polycyclic (e.g. bicyclic). A polycyclic heterocyclyl may for example contain fused rings. In a bicyclic heterocyclyl group there may be one or more heteroatoms in each ring, or heteroatoms only in one of the rings. A heteroatom may be N, O, or S. Heterocyclyl groups containing a suitable nitrogen atom include the corresponding N-oxides. In one example, the heterocyclyl group is of three to ten atoms (i.e. 3-10-membered heterocyclyl). In one example, the heterocyclyl group is of three to seven atoms (i.e. 3-7-membered heterocyclyl). Examples of monocyclic non-aromatic heterocyclyl groups include aziridinyl, azetidinyl, pyrrolidinyl, imidazolidinyl, pyrazolidinyl, piperidinyl, piperazinyl, tetrahydrofuranyl, tetrahydropyranyl, morpholinyl, thiomorpholinyl and azepanyl. Examples of bicyclic heterocyclyl groups in which one of the rings is non-aromatic include dihydrobenzofuranyl, indanyl, indolinyl, isoindolinyl, tetrahydroisoquinolinyl, tetrahydroquinolyl, and benzoazepanyl. Examples of monocyclic aromatic heterocyclyl groups (also referred to as monocyclic heteroaryl groups) include furanyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl. Examples of bicyclic aromatic heterocyclyl groups (also referred to as bicyclic heteroaryl groups) include quinoxalinyl, quinazolinyl, pyridopyrazinyl, benzoxazolyl, benzothiophenyl, benzimidazolyl, naphthyridinyl, quinolinyl, benzofuranyl, indolyl, benzothiazolyl, oxazolyl[4,5-b]pyridyl, pyridopyrimidinyl, isoquinolinyl, and benzohydroxazole.

### Depression

The subject matter of the present disclosure is predicated in part on the surprising discovery that a compound of Formula I can be used for treating or preventing depression in subjects resistant to antidepressant treatments and/or treating or preventing depression associated cognitive impairment in subjects resistant to antidepressant treatments. Accordingly, in one aspect, there is provided a method of treating or preventing depression in subjects resistant to antidepressant treatments and/or treating or preventing depression associated cognitive impairment in subjects resistant to antidepressant treatments.

One skilled in the art will recognize that wherein the present disclosure is directed to methods of prevention, a subject in need thereof will include any subject or patient (e.g. a human) who has experienced or exhibited at least one symptom of the disorder, disease or condition to be prevented. Further, a subject in need thereof may additionally be a subject who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

### Anti-Depressant Resistance

As used herein, the term “depression” includes major depressive disorder (MDD), unipolar depression, depression with anxious distress, bipolar depression and dysthymia. In one example, the depression is MDD. The depression may be defined as treatment resistant depression, such as treatment resistant MDD.

As used herein, the term “treatment resistant depression” or “resistant to antidepressant treatment” may be defined as depression (e.g. MDD) that does not respond to adequate courses of adequate treatments of one or more anti-depressants, for example for at least two antidepressants. One skilled in the art will recognize that a failure to respond to an adequate course of a given antidepressant may be readily determined retrospectively or prospectively.

As used herein, unless the context requires otherwise, the term “antidepressant” means any pharmaceutical agent which can be used to treat depression. Suitable examples include, but are not limited to mono-amine oxidase inhibitors such as phenelzine, tranylcypromine, moclobemide, and the like; tricyclics such as imipramine, amitriptyline, desipramine, nortriptyline, doxepin, protriptyline, trimipramine, chlomipramine, amoxapine, and the like; tetracyclics such as maprotiline, and the like; non-cyclics such as nomifensine, and the like; triazolopyridines such as trazodone, and the like; serotonin reuptake inhibitors such as fluoxetine, sertraline, paroxetine, citalopram, citolapram, escitolapram, fluvoxamine, and the like; serotonin receptor antagonists such as nefazadone, and the like; serotonin noradrenergic reuptake inhibitors such as venlafaxine, milnacipran, desvenlafaxine, duloxetine and the like; noradrenergic and specific serotonergic agents such as mirtazapine, and the like; noradrenaline reuptake inhibitors such as reboxetine, edivoxetine and the like; atypical antidepressants such as bupropion, and the like; natural products such as Kava-Kava, St. John's Wort, and the like; dietary supplements such as s-adenosylmethionine, and the like; and neuropeptides such as thyrotropin-releasing hormone and the like; compounds targeting neuropeptide receptors such as neurokinin receptor antagonists and the like; and hormones such as triiodothyronine, and the like.

Therapeutically effective dosage levels and dosage regimens for antidepressants (for example, mono-amine oxidase inhibitors, tricyclics, serotonin reuptake inhibitors, serotonin noradrenergic reuptake inhibitors, noradrenergic and specific serotonergic agents, noradrenaline reuptake inhibitor, natural products, dietary supplements, neuropeptides, compounds targeting neuropeptide receptors, hormones and other pharmaceutical agents disclosed herein), may be readily determined by one of ordinary skill in the art. For example, therapeutic dosage amounts and regimens for pharmaceutical agents approved for sale are publicly available, for example as listed on packaging labels, in standard dosage guidelines, in standard dosage references such as the Physician's Desk Reference or other sources.

In one example, the subject resistant to antidepressant treatment is resistant to one or more antidepressants selected from the group consisting of serotonin specific reuptake inhibitors (SSRI), noradrenaline reuptake inhibitors (NARI), noradrenaline and specific serotonergic antidepressants (NaSSA), melatonergic agonists (MA), serotonin and noradrenaline reuptake inhibitors (SNRI), reversible inhibitors of monoamine oxidase (RIMAs), tricyclic antidepressants (TCA), mono-amine oxidase inhibitors (MAOI), and serotonin modulators (SM).

In another example, the subject resistant to antidepressant treatment is resistant to one or more antidepressants selected from the group consisting of imipramine, amitriptyline, dothiepin, desipramine, nortriptyline, doxepin, protriptyline, trimipramine, maprotiline, amoxapine, trazodone, bupropion, chlomipramine, fluoxetine, citalopram, sertraline, paroxetine, tianeptine, nefazadone, venlafaxine, desvenlafaxine, duloxetine, reboxetine, mirtazapine, phenelzine, tranylcypromine, moclobemide, reboxetine, mianserin, agomelatine, and vortioxetine.

In another example, the subject resistant to antidepressant treatment is resistant to one or more antidepressants selected from the group consisting of selective serotonin reuptake inhibitors (SSRI). The SSRI may be selected from one or more of citalopram, escitalopram, fluoxetine, fluvoxamine, paroxetine, sertraline, dapoxetine, indalpine, zimelidine, alaproclate, centpropazine, cericlamine, femoxetine, ifoxetine, omiloxetine, panuramine, pirandamine, and seproxetine. For example, the SSRI can be selected from one or more of citalopram, escitalopram, fluoxetine, fluvoxamine, paroxetine, sertraline.

In another example, the subject resistant to antidepressant treatment is resistant to one or more antidepressants selected from the group consisting of noradrenaline reuptake inhibitors (NARI). In one example, the NARI is reboxetine.

In another example, the subject resistant to antidepressant treatment is resistant to one or more antidepressants selected from the group consisting of noradrenaline and specific serotonergic antidepressants (NaSSA). In one example, the NaSSA is selected from mirtazapine and/or mianserin.

In another example, the subject resistant to antidepressant treatment is resistant to one or more antidepressants selected from the group consisting of melatonergic agonists (MA). In one example, the MA is agomelatine.

In another example, the subject resistant to antidepressant treatment is resistant to one or more antidepressants selected from the group consisting of serotonin and noradrenaline reuptake inhibitors (SNRI). In one example, the SNRI is selected from one or more of atomoxetine, desvenlafaxine, duloxetine, levomilnacipran, milnacipran, sibutramine, tramadol, and venlafaxine.

In another example, the subject resistant to antidepressant treatment is resistant to one or more antidepressants selected from the group consisting of reversible inhibitors of monoamine oxidase (RIMAs). In one example, the RIMA is moclobemide.

In another example, the subject resistant to antidepressant treatment is resistant to one or more antidepressants selected from the group consisting of tricyclic antidepressants (TCA). In one example, the TCA is selected from one or more of amitriptyline, clomipramine, dothiepin, imipramine, nortriptyline, and doxepin.

In another example, the subject resistant to antidepressant treatment is resistant to one or more antidepressants selected from the group consisting of mono-amine oxidase inhibitors (MAOI). In one example, the MAOIs is selected from phenelzine and/or tranylcypromine.

In another example, the subject resistant to antidepressant treatment is resistant to one or more antidepressants selected from the group consisting of serotonin modulators (SM). In one example, the SM is vortioxetine.

In another example, the subject resistant to antidepressant treatment is a subject resistant to treatment of major depressive disorder (MDD).

It will be appreciated that there are well known methods for diagnosing depression (e.g. MDD) in a subject. In one example, depression or MDD is diagnosed by identifying a subject using a Hamilton Depression Rating Scale (HAM-D or HDRS). The HAM-D scale involves 17 items with a maximum rating of 54 indicating severe depression. In some examples, the HAM-D rating may be at least 15, 16, 17, 18, 19, 20, 21, 22, 23, 24, 25, 26, 27, 28, 29, or 30. In one example, the HAM-D is at least 17. In another example, depression or MDD is diagnosed using the Diagnostic Statistical Manual of Mental Disorders (DSM-5) criteria (see American Psychiatric Association's Diagnostic Statistical Manual of Mental Disorders, Fifth Edition). The DSM-IV or DSM-V criteria may also be obtained using a Mini International Neuropyschiatric Interview (MINI) (Lecrubier at al, Eur Psychiatry, 1997, 12, pp. 224-231). Further details may be obtained online at https://harmresearch.org/mini-international-neuropsychiatric-interview-mini/), which is incorporated herein by reference thereto.

It will be appreciated that the administration of a compound of Formula I, or a pharmaceutically acceptable salt, solvate or prodrug thereof, can provide an improvement of depression in the treated subject relative to a non-treated subject.

An improvement of depression in the treated subject may be determined using the Montgomery and Asberg Depression Rating Scale (MADRS), which involves a clinical interview moving from broadly phrased questions about symptoms to more detailed ones which allow a precise rating of severity. The rater decides whether the rating lies on the defined scale steps (0, 2, 4, 6) or between them (1,3,5). The questions are directed to apparent sadness, reported sadness, inner tension, reduced sleep, reduced appetite, concentration difficulties, lassitude, inability to fell, pessimistic thoughts, and suicidal thoughts. Further details can be obtained at Quilty et al, International Journal of Methods in Psychiatric Research, Int. J. Methods Psychiatr. Res. 22(3): 175-184 (2013) (wileyonlinelibrary.com) DOI: 10.1002/mpr.1388. The MADRS produces a rating of 0-60, with higher scores reflecting greater severity of depression. In some examples, the MADRS rating is at least 10, 15, 20, 25, 30, 35, 40, 45, or 50. In some examples, the method provides a reduction in the MADRS rating of the treated subject by at least about 1, 2, 3, 4, 5, 10, 15, 20, 25, 30, 35, 40, 45, or 50. In some examples, the method provides an improvement in the MADRS rating of the treated subject by at least about 10%, 20%, 30%, 40%, 50%, 60%, 70%, 80%, or 90%.

### Cognitive Impairment

The subject matter of the present disclosure is predicated in part on the surprising discovery that a compound of Formula I can be used for treating or preventing depression in subjects resistant to antidepressant treatments and/or treating or preventing depression associated cognitive impairment in subjects resistant to antidepressant treatments. Accordingly, in one aspect, there is provided a method of treating or preventing depression associated cognitive impairment in subjects resistant to antidepressant treatments.

The depression associated cognitive impairment in a subject resistant to antidepressant treatment may be determined using the Wechsler Adult Intelligence Scale (WAIS) scale, for example WAIS-IV released in 2008. WAIS-IV is composed of 10 core subtests and five supplemental subtests, with the 10 core subtests comprising the Full Scale IQ. With the new WAIS-IV, the verbal/performance subscales from previous versions were removed and replaced by the index scores. The General Ability Index (GAI) was included, which consists of the Similarities, Vocabulary and Information subtests from the Verbal Comprehension Index and the Block Design, Matrix Reasoning and Visual Puzzles subtests from the Perceptual Reasoning Index. The GAI is clinically useful because it can be used as a measure of cognitive abilities that are less vulnerable to impairment. Wechsler, David (1939) entitled “The measurement of adult intelligence. Baltimore: Williams & Wilkins. p. 229. “Wechsler Adult Intelligence Scale—Revised”. http://www.cps.nova.edu/-cpphelp/WAIS-R.html.

There are four index scores representing major components of intelligence:
Verbal Comprehension Index (VCI)
Perceptual Reasoning Index (PRI)
Working Memory Index (WMI)
Processing Speed Index (PSI)

In some examples, the subject has at least 0.1, 0.5, 1, 2, or 5, SD impairment on WAIS Coding Test relative to age, education and gender.

In other examples, the depression associated cognitive impairment in a subject resistant to antidepressant treatment may be determined using Cogstate Neuropyschological Testing Battery criteria, Hopkins Verbal Learning Test (or Hopkins Verbal Learning Test Revised), Controlled Word Association Test, and Trail Making A and B Tests, Category Fluency Test, Letter Fluency Test, Patient Global Impression of Severity (PGI-S) test, and the Patient Global Impression of Improvement (PGI-I) test.

In other examples, the depression associated cognitive impairment in a subject resistant to antidepressant treatment may be determined using Cogstate Neuropyschological Testing Battery criteria, Hopkins Verbal Learning Test (or Hopkins Verbal Learning Test Revised), Controlled Word Association Test, and Trail Making A and B Tests.

In some examples, the improvement is by showing a change in baseline in Executive Function and/or Episodic Memory. A change in baseline Executive Function and/or Episodic Memory may be assessed by reference to any one of the tests or criteria described herein, or as a composite of one or more if the tests or criteria described herein. In one example, the improvement in Executive Function is assessed according to a composite of the performance of the treated subject in the One Back Test, Category Fluency Test [CFT], Letter Fluency Test [LFT], and International Digit Symbol Substitution Test-Symbols. In another example, the improvement in Executive Function is assessed according to a composite of the performance of the treated subject in the One Card Learning and Hopkins Verbal Learning Test—Revised [HVLT-R], (Immediate Recall only).

In one example, the depression associated cognitive impairment in a subject resistant to antidepressant treatment may be determined using Hopkins Verbal Learning Test (HVLT) or the Hopkins Verbal Learning Test Revised (HVLT-R). The HVLT-R is a measure of verbal episodic memory that consists of 3 initial learning trials, a delayed recall trial and a yes/no delayed recognition trial. The HVLT-R comes in 6 alternative and parallel forms, thereby reducing the potential of a practice effect. In some. In some examples, the method provides an improvement in the HVLT or HVLT-R rating of the treated subject. In some examples, the method provides an improvement in the HVLT or HVLT-R rating of the treated subject by at least about 10%, 20%, 30%, 40%, 50%, 60%, 70%, 80%, or 90%.

In one example, the depression associated cognitive impairment in a subject resistant to antidepressant treatment may be determined using the Trail Making A and B test. The Trail Making A and B test is a paper-based test used to assess performance in speed of processing and executive function. Part A of the test assesses complex attention and requires a participant to connect randomly positioned numbered circles as quickly as possible. Part B assesses executive function and requires connection of circled letters and numbers in alternating numeric and alphabetic sequence (ie, 1-A-2-B, etc.) as quickly as possible, alternating between numbers and letters. In some examples, the method provides an improvement in the Trail Making A and B performance of the treated subject. In some examples, the method provides an improvement in the Trail Making A and B performance of the treated subject by at least about 10%, 20%, 30%, 40%, 50%, 60%, 70%, 80%, or 90%.

In one example, the depression associated cognitive impairment in a subject resistant to antidepressant treatment may be determined using Category Fluency Test (CFT). In one example, the depression associated cognitive impairment in a subject resistant to antidepressant treatment may be determined using Category Fluency Test (CFT). Letter Fluency Test (LFT). The CFT and LFT measure performance in language and executive function domains, and are paper-based verbal fluency tests. The CFT measures spontaneous production of words that are exemplars of different categories (such as animals, fruits, and vegetables) in 1 minute. The LFT measures spontaneous productions of words beginning with a designated letter (3 different letters are assessed at each applicable trial visit, such as F, A, and S) in 1 minute. In some examples, the method provides an improvement in the CFT score of the treated subject. In some examples, the method provides an improvement in the CFT score of the treated subject by at least about 5%, 10%, 20%, 30%, 40%, 50%, 60%, 70%, 80%, or 90%. In some examples, the method provides an improvement in the LFT score of the treated subject. In some examples, the method provides an improvement in the LFT score of the treated subject by at least about 5%, 10%, 20%, 30%, 40%, 50%, 60%, 70%, 80%, or 90%.

In one example, the depression associated cognitive impairment in a subject resistant to antidepressant treatment may be determined using Patient Global Impression of Severity (PGI-S) and Patient Global Impression of Improvement (PGI-I). PGI-S and PGI-I enables a participant to provide an assessment of their perception of the severity of their symptoms and any improvements observed during the trial. Participants are prompted to describe their personal global perception of their depression and cognitive impairment. For the PGI-S, the participant will rate the severity of their symptoms on a seven-point scale: 1, not present; 2, very mild; 3, mild; 4, moderate; 5, moderately severe; 6, severe; 7, extremely severe. The 7-point scale for the PGI-I is rated as 1, very much improved; 2, much improved; 3, minimally improved; 4, no change; 5, minimally worse; 6, much worse; or 7, very much worse. In some examples, the method provides an improvement in the PGI-S score of the treated subject. In some examples, the method provides an improvement in the PGI-S score of the treated subject by at least about 1, 2, 3, 4, 5, or 6. In some examples, the method provides an improvement in the PGI-I score of the treated subject. In some examples, the method provides an improvement in the PGI-I score of the treated subject by at least about 1, 2, 3, 4, 5, or 6.

In one example, the depression associated cognitive impairment in a subject resistant to antidepressant treatment may be determined using Cogstate Neuropyschological Testing Battery criteria.

As used herein, “cognitive impairment” refers to the deterioration, or loss, of cognitive ability relative to the subject. Such deterioration, or loss, of cognitive ability, may be evident in an otherwise cognitively healthy subject.

Other medical conditions that may be associated with cognitive impairment or decline include medical conditions that primarily affect, or are highly associated with, the central nervous system (CNS). In some embodiments, the subject is not suffering from another medical condition that is associated with the central nervous system (CNS), for example Alzheimer's disease (AD).

In some embodiments, the subject may not have a disorder associated with a substantially elevated level of cortisol, such as dementia. Dementias include, but are not limited to Alzheimer's disease, multi-infarct dementia, dementia with Lewy bodies, fronto-temporal dementia (including Pick's disease), progressive supranuclear palsy, Korsakoff's syndrome, Biswanger's disease, HIV-associated dementia, Creutzfeldt-Jakob disease (CJD), multiple sclerosis, motor neurone disease, Parkinson's disease, Huntington's disease, Niemann-Pick disease type C, normal pressure hydrocephalus, and Down's syndrome.

The subject may have a cortisol level (in nmol/L) of less than about 450, 440, 430, 420, 410, 400, 390, 380, 370, 360, 250, 340, 330, 320, 310, 300, 290, 280, 270, 260, 250, 240, 230, 220, 210, or 200. The subject may have a cortisol level (in nmol/L) of at least about 150, 160, 170, 180, 190, 200, 210, 220, 230, 240, 250, 260, 270, 280, 290, 300, 310, 320, 330, 340, or 350. The subject may have a cortisol level (in nmol/L) in a range provided by any two of these upper and/or lower levels. For example, the subject may have a cortisol level (in nmol/L) in a range between about 150 and 400, 170 and 350, or 200 and 330. The cortisol level may be measured from the plasma or serum of a subject. The cortisol level may represent an average daily or weekly measurement.

### Age and Gender of Subjects

It is understood that cognitive decline varies amongst different subjects. In some embodiments, the age (in years) of the subject is at least about 5, 10, 15, 18, 20, 30, 40, 50, 60, 70, 75, 80, 90, or 100. In some embodiments, the age of the subject (in years) is less than about 90, 80, 75, 70, 60, 50, 40, 30, 20, 18, or 10. The age of the subject may be in a range between any two of these upper and/or lower values.

Subjects with Increased Risk of Depression and Associated Cognitive Impairment

It will also be understood that the method disclosed herein may find application in the prevention and/or reduction of cognitive impairment in a subject that is at increased risk of antidepressant resistant depression and associated cognitive impairment. “Increased risk” is used herein to refer to a subject that may have a higher likelihood of experiencing antidepressant resistant depression and associated cognitive impairment. In some embodiments, the subject has an increased risk of antidepressant resistant depression. In some embodiments, the subject has an increased risk of antidepressant resistant cognitive impairment. In some embodiments, the subject has an increased risk of experiencing severe antidepressant resistant depression and associated cognitive impairment.

The person skilled in the art will appreciate that there are a number of factors that may result in a subject having an increased risk of antidepressant resistant depression and associated cognitive impairment. Factors that may result in a subject having an increased risk include, but are not limited to, the presence of particular genetic markers, a family history of antidepressant resistant depression and associated cognitive impairment, suffering from particular medical conditions associated with antidepressant resistant depression and associated cognitive impairment, and/or environmental factors that are associated with antidepressant resistant depression and associated cognitive impairment, and/or societal factors that are associated with antidepressant resistant depression and associated cognitive impairment, and/or external factors that are associated with antidepressant resistant depression and associated cognitive impairment.

### Enhancement of Cognitive Ability

It will also be understood that the method disclosed herein may be beneficial in treating or preventing cognitive impairment in a subject having antidepressant resistant depression and/or associated cognitive impairment. As used herein, “treating” refers to an increase in cognitive ability compared to a subject's previous ability (i.e., before being administered a compound of Formula I, or a pharmaceutically acceptable salt, solvate or prodrug thereof). In some embodiments, the subject experiences improved cognitive ability. The enhancement of cognitive ability may be experienced in relation to one or more facets of cognition including, for example, psychomotor function, visual attention, learning and memory, working memory, and visual associate memory. Such cognitive abilities may be measured using various techniques known in the field, such as those described below (e.g. Cogstate Battery Tests). In some embodiments, a subject experiences enhanced psychomotor function. In some embodiments, a subject experiences enhanced visual attention. In some embodiments, a subject experiences enhanced learning and memory. In some embodiments, a subject experiences enhanced working memory. In some embodiments, a subject experiences enhanced visual associate memory.

### Methods of Assessing Cognitive Ability

There are several tests through which the cognitive ability of a subject may be assessed. Accordingly, based on the results of these tests, it is possible to assess a subject's cognitive decline.

The person skilled in the art will appreciate that there exists a variety of tests that can be employed to accurately assess the cognitive state, and similarly the cognitive decline, of a subject. Such tests include, but are not limited to, Cogstate Battery Tests, Cambridge Neuropsychological Test Automated Battery (CANTAB), Intelligence Quotient (IQ) Test, Kohs Block Design Test, Miller Analogies Test, Otis-Lennon School Ability Test (OLSAT), Raven's Progressive Matrices, Stanford-Binet Intelligence Scales, Wechsler Intelligence Scale for Children (WISC), Wonderlic Test, Porteus Maze Test, Pimsleur Language Aptitude Battery, Knox Cubes, Draw-a-Person Test, Mini-Mental State Exam (MMSE), the Alzheimer's Disease Assessment Scale-Cognitive Subscale (ADAS-Cog, ADCOMs, Rey Auditory Visual Learning Test (RAVLT), NTB and NPI), and CDR Computerized Assessment System. Cogstate Battery Tests include, for example, Detection test, Identification test, One Card Learning test, One Back Working Memory test, the Continuous Paired Associate Learning test, and the International Digit Symbol Substitution Test-Symbols. Each test is designed to assess various parameters that may be attributed to the cognitive ability of a subject. Accordingly, in some embodiments the administration of a compound of Formula I, or a pharmaceutically acceptable salt, solvate or prodrug thereof, provides improved cognitive testing (Cogstate Test Battery) of the treated subject relative to a non-treated subject. An improvement in cognitive testing, as determined by the Cogstate Test Battery results, may also be considered from a summation of each individual test results (e.g., Detection test, Identification test, One Card Learning test, One Back Working Memory test, International Digit Symbol Substitution Test, and the Continuous Paired Associate Learning test).

In some examples, any one or more cognitive tests measuring reaction time improvement may provide an improvement of at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, any one or more cognitive tests may provide a “p value” probability measurement from a treated subject group of less than about 0.5, 0.4, 0.3, 0.2, 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, or 0.001. In some examples, the cognitive testing may provide a Cohen's “d-value” standardised difference between two means (i.e. treated and untreated subject) of at least about 0.1, 0.2, 0.3, 0.4, 0.5, 0.6, 0.7, or 0.8. In some examples, the cognitive testing may provide a “p-value” and “d-value” according to any combination of individual embodiments thereof as described herein.

In some embodiments, the administration of a compound of Formula I, or a pharmaceutically acceptable salt, solvate or prodrug thereof, provides an improvement of psychomotor function of the treated subject relative to a non-treated subject. Psychomotor function is evidenced by an improvement in simple reaction time, and is measured in speed of performance (Log₁₀ milliseconds). As will be appreciated by the person skilled in the art, psychomotor function is critical in a subject's ability to execute both gross and fine motor skills. Accordingly, psychomotor function is an essential component of physical skills including, for example, movement, coordination, manipulation, dexterity, grace, strength, and speed. It is therefore foreseeable that the method described herein may find particular application in improving psychomotor function in a cognitively healthy subject. In some embodiments, the administration of a compound of Formula I, or a pharmaceutically acceptable salt, solvate or prodrug thereof, provides an improvement of psychomotor function in a cognitively healthy subject. In some embodiments, the administration of a compound of Formula I, or a pharmaceutically acceptable salt, solvate or prodrug thereof, provides an improvement in physical skill of a cognitively healthy subject. In some embodiments, the administration of a compound of Formula I, or a pharmaceutically acceptable salt, solvate or prodrug thereof, provides an improvement of gross motor skills in a cognitively healthy subject. In some embodiments, the administration of a compound of Formula I, or a pharmaceutically acceptable salt, solvate or prodrug thereof, provides an improvement of fine motor skills in a cognitively healthy subject.

From here, it is foreseeable that the method described herein may also find particular application in assisting a subject in regaining psychomotor function. A loss of psychomotor function may result for any number of reasons including, but not limited to, accident and/or injury, a stroke, or other medical condition. Such a loss of psychomotor skills may affect any one or more areas of the subject's body including, but not limited to, the limbs. Accordingly, the administration of a compound of Formula I, or a pharmaceutically acceptable salt, solvate or prodrug thereof, may assist to restore psychomotor function. The term “restore psychomotor function” is used herein to mean a return, to some extent, of previous motor function.

In some embodiments, the administration of a compound of Formula I, or a pharmaceutically acceptable salt, solvate or prodrug thereof, provides an improvement of psychomotor function speed of performance. The psychomotor function speed improvement may be measured between the treated subjects relative to non-treated subjects. In some examples, a psychomotor function speed improvement may be provided by a log 10 reaction time (milliseconds) of less than about 2.60. 2.59, 2.58, 2.57, 2.56, 2.55, 2.54, 2.53, 2.52, 2.51, or 2.50. In some examples, a psychomotor function speed improvement may be at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, the psychomotor function testing has a p value probability measurement from a treated subject group of less than about 0.5, 0.4, 0.3, 0.2, 0.1, or 0.09. In some examples, the psychomotor function testing may provide a Cohen's d-value standardised difference between two means (i.e. treated and untreated subject) of at least about 0.1, 0.2, 0.3, 0.4, 0.5, 0.6, or 0.7. In some examples, the psychomotor function testing may provide a “p-value” and “d-value” according to any combination of individual embodiments thereof, such as a p-value less than about 0.09 with a d-value effect size of greater than about 0.7.

In some embodiments, the administration of a compound of Formula I, or a pharmaceutically acceptable salt, solvate or prodrug thereof, provides an improvement of visual attention of the treated subject relative to a non-treated subject. Visual attention is evidenced by an improvement in choice retention time, and is measured in speed of performance (Log₁₀ milliseconds). In some embodiments, the improvement in visual attention is evidenced by an improvement in choice retention time. In some embodiments, the improvement in visual attention is assessed by the Cogstate Identification Test. In some embodiments, the administration of a compound of Formula I, or a pharmaceutically acceptable salt, solvate or prodrug thereof, provides an improvement of visual attention speed of performance of the treated subject relative to a non-treated subject. The visual attention speed improvement may be measured between the treated subjects relative to non-treated subjects. In some examples, a visual attention speed improvement may be provided by a log 10 reaction time (milliseconds) of less than about 2.80, 2.79, 2.78, 2.77, 2.76, 2.75, 2.74, 2.73, 2.72, 2.71, or 2.70. In some examples, a visual attention speed improvement may be at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, the visual attention testing has a p value probability measurement from a treated subject group of less than about 0.5, 0.4, 0.3, 0.2, 0.1, or 0.09, 0.08, 0.07, 0.06, or 0.05. In some examples, the visual attention may provide a Cohen's d-value standardised difference between two means (i.e. treated and untreated subject) of at least about 0.1, 0.2, 0.3, 0.4, 0.5, or 0.6. In some examples, the visual attention testing may provide a “p-value” and “d-value” according to any combination of individual embodiments thereof, such as a p-value less than about 0.05 with a d-value effect size of greater than about 0.6.

In some embodiments, the administration of a compound of Formula I, or a pharmaceutically acceptable salt, solvate or prodrug thereof, provides an improvement of learning and memory of the treated subject relative to a non-treated subject. Learning and memory is evidenced by an improvement in visual recognition learning, and is measured in accuracy of performance (arcsine proportion correct). In some embodiments, the improvement in learning and memory is evidenced by an improvement in visual recognition learning. In some embodiments, the improvement in learning and memory is assessed by the Cogstate One Card Learning Test. In some embodiments, the administration of a compound of Formula I, or a pharmaceutically acceptable salt, solvate or prodrug thereof, provides an improvement of learning and memory accuracy of proportion (i.e., arcsine proportion correct) of at least about X % of the treated subject relative to a non-treated subject. The learning and memory accuracy improvement may be measured between the treated subjects relative to non-treated subjects. In some examples, a learning and memory accuracy testing may be provided by an arcsine accuracy of at least about 1.0, 1.01, 1.02, 1.03, 1.04, 1.05, 1.06, 1.07, 1.08, 1.09. or 1.10. In some examples, the learning and memory accuracy testing has a p value probability measurement from a treated subject group of less than about 0.9. In some examples, the visual attention may provide a Cohen's d-value standardised difference between two means (i.e. treated and untreated subject) of at least about 0.1, 0.15, or 0.19. In some examples, the visual attention testing may provide a “p-value” and “d-value” according to any combination of individual embodiments thereof, such as a p-value less than about 0.9 with a d-value effect size of greater than about 0.15.

In some embodiments, the administration of a compound of Formula I, or a pharmaceutically acceptable salt, solvate or prodrug thereof, provides an improvement of working memory of the treated subject relative to a non-treated subject. In some embodiments, the improvement in working memory is assessed by the Cogstate One Back Working Memory Test, and is measured in number of errors. In some embodiments, the administration of a compound of Formula I, or a pharmaceutically acceptable salt, solvate or prodrug thereof, provides an improvement of working memory number of errors of at least about X % of the treated subject relative to a non-treated subject. The working memory improvement may be measured between the treated subjects relative to non-treated subjects. In some examples, a working memory test may be provided by a log 10 reaction time of less than about 2.90, 2.89, 2.88, 2.87, 2.86, 2.85, 2.84, 2.83, 2.82, 2.81, or 2.80. In some examples, a working memory test improvement may be at least about (log 10, milliseconds) 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, or 0.20. In some examples, the working memory testing has a p value probability measurement from a treated subject group of less than about 0.5, 0.4, 0.3, 0.2, 0.1, or 0.09, 0.08, 0.07, 0.06, 0.05, 0.04, 0.03, 0.02, or 0.01. In some examples, the working memory testing may provide a Cohen's d-value standardised difference between two means (i.e. treated and untreated subject) of at least about 0.1, 0.2, 0.3, 0.4, 0.5, 0.6, 0.7, or 0.8. In some examples, the visual attention testing may provide a “p-value” and “d-value” according to any combination of individual embodiments thereof, such as a p-value less than about 0.01 with a d-value effect size of greater than about 0.8.

In some embodiments, the administration of a compound of Formula I, or a pharmaceutically acceptable salt, solvate or prodrug thereof, provides an improvement of visual associate memory of the treated subject relative to a non-treated subject. In some embodiments, visual associate memory is assessed by the Cogstate Continuous Paired Associate Learning Test, and is measured in number of errors. The visual associate memory improvement may be measured between the treated subjects relative to non-treated subjects. In some examples, a visual associate memory testing may be provided by an error score out of 30 of less than 25, 24, 23, 22, 21, 20, 19, 18, 17, 16, 15, 14, 13, 12, 11, or 10. In some examples, a visual associate memory testing may be provided by an improvement in error score of at least 1, 2, 3, 4, 5, 6, 7, 8, 9, 10, 11, 12, 13, 14, 15, 16, 17,18, 19, 20, 21, 22, 23, 24, 25, 26, 27, 28, 29, or 30. In some examples, the visual associate memory testing has a p value probability measurement from a treated subject group of less than about 0.5. In some examples, the visual associate memory testing may provide a Cohen's d-value standardised difference between two means (i.e. treated and untreated subject) of at least about 0.1, 0.2, 0.3, or 0.4. In some examples, the visual associate memory testing may provide a “p-value” and “d-value” according to any combination of individual embodiments thereof, such as a p-value less than about 0.5 with a d-value effect size of greater than about 0.4.

### Compound of Formula I

It has been surprisingly found that a compound of Formula I can be used for treating or preventing depression in subjects resistant to antidepressant treatments and/or treating or preventing depression associated cognitive impairment in subjects resistant to antidepressant treatments. Embodiments of the methods and uses described herein comprise administering a compound of Formula I, or a pharmaceutically acceptable salt, solvate or prodrug thereof.

As used herein, a compound of Formula I has the following chemical structure:

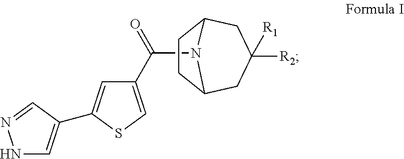

wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR³, —NR³R⁴, —COR³, —CO₂R³, —CONR³R⁴, —NR³COR⁴, —SO₂R³, —SO₂NR³R⁴, and —NR³SO₂R⁴.

In some embodiments, R¹ is hydrogen. In some embodiments, R¹ is halogen. In some embodiments, R¹ is chlorine. In some embodiments, R¹ is fluorine. In some embodiments, R¹ is bromine. In some embodiments, R¹ is iodine. In some embodiments, R¹ is C₁₋₆alkyl. In some embodiments, R¹ is —O—C₁₋₆alkyl. In some embodiments, R¹ is C₁₋₆haloalkyl. In some embodiments, R¹ is —O—C₁₋₆haloalkyl. In some embodiments, R¹ is C₂₋₆alkenyl. In some embodiments, R¹ is C₂₋₆alkynyl. In some embodiments, R¹ is 3-10-membered carbocyclyl. In some embodiments, R¹ is a 6-membered carbocyclyl. In some embodiments, R¹ is a 5-membered carbocyclyl. In some embodiments, R¹ is 3-10-membered heterocyclyl. In some embodiments, R¹ is a 6-membered heterocyclyl. In some embodiments, R¹ is a 5-membered heterocyclyl. In some embodiments, R¹ is —CN. In some embodiments, R¹ is —CF₃. In some embodiments, R¹ is —OR³. In some embodiments, R¹ is —SR³. In some embodiments, R¹ is —NR³R⁴. In some embodiments, R¹ is —COR₃. In some embodiments, R¹ is —CO₂R³. In some embodiments, R¹ is —CONR³R⁴. In some embodiments, R¹ is —NR³COR⁴. In some embodiments, R¹ is —SO₂R³. In some embodiments, R¹ is —SO₂NR³R⁴. In some embodiments, R¹ is —NR³SO₂R⁴.

In some embodiments, R² is hydrogen. In some embodiments, R² is halogen. In some embodiments, R² is chlorine. In some embodiments, R² is fluorine. In some embodiments, R² is bromine. In some embodiments, R² is iodine. In some embodiments, R² is C₁₋₆alkyl. In some embodiments, R² is —O—C₁₋₆alkyl. In some embodiments, R² is C₁₋₆haloalkyl. In some embodiments, R² is —O—C₁₋₆haloalkyl. In some embodiments, R² is C₂₋₆alkenyl. In some embodiments, R² is C₂₋₆alkynyl. In some embodiments, R² is 3-10-membered carbocyclyl. In some embodiments, R² is a 6-membered carbocyclyl. In some embodiments, R² is a 5-membered carbocyclyl. In some embodiments, R² is 3-10-membered heterocyclyl. In some embodiments, R² is a 6-membered heterocyclyl. In some embodiments, R² is a 5-membered heterocyclyl. In some embodiments, R² is —CN. In some embodiments, R² is —CF₃. In some embodiments, R² is —OR³. In some embodiments, R² is —SR³. In some embodiments, R² is —NR³R⁴. In some embodiments, R² is —COR³. In some embodiments, R² is —CO₂R³. In some embodiments, R² is —CONR³R⁴. In some embodiments, R² is —NR³COR⁴. In some embodiments, R² is —SO₂R³. In some embodiments, R² is —SO₂NR³R⁴. In some embodiments, R² is —NR³SO₂R⁴.

If present, each 3-10-membered carbocyclyl and 3-10-membered heterocyclyl may be further substituted with one or more substituents selected from the group consisting of hydrogen, halogen, C₁₋₆alkyl, —O—C₁₋₆alkyl, C₁₋₆haloalkyl, —O—C₁₋₆ haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶.

In some embodiments, the 3-10-membered carbocyclyl is substituted with one or more halogen substituents. In some embodiments, the 3-10-membered carbocyclyl is substituted with one or more C₁₋₆alkyl substituents. In some embodiments, the 3-10-membered carbocyclyl is substituted with one or more —O—C₁₋₆alkyl substituents. In some embodiments, the 3-10-membered carbocyclyl is substituted with one or more C₁₋₆ haloalkyl substituents. In some embodiments, the 3-10-membered carbocyclyl is substituted with one or more —O—C₁₋₆haloalkyl substituents. In some embodiments, the 3-10-membered carbocyclyl is substituted with one or more C₂₋₆alkenyl substituents. In some embodiments, the 3-10-membered carbocyclyl is substituted with one or more C₂-6alkynyl substituents. In some embodiments, the 3-10-membered carbocyclyl is substituted with one or more —CN substituents. In some embodiments, the 3-10-membered carbocyclyl is substituted with one or more —CF₃ substituents. In some embodiments, the 3-10-membered carbocyclyl is substituted with one or more —OR⁵ substituents. In some embodiments, the 3-10-membered carbocyclyl is substituted with one or more —SR⁵ substituents. In some embodiments, the 3-10-membered carbocyclyl is substituted with one or more —COR⁵ substituents. In some embodiments, the 3-10-membered carbocyclyl is substituted with one or more halogen substituents. In some embodiments, the 3-10-membered carbocyclyl is substituted with one or more —CO₂R⁵ substituents. In some embodiments, the 3-10-membered carbocyclyl is substituted with one or more —CONR⁵R⁶ substituents. In some embodiments, the 3-10-membered carbocyclyl is substituted with one or more —NR⁵COR⁶ substituents. In some embodiments, the 3-10-membered carbocyclyl is substituted with one or more —SO₂R⁵ substituents. In some embodiments, the 3-10-membered carbocyclyl is substituted with one or more —SO₂NR⁵R⁶ substituents. In some embodiments, the 3-10-membered carbocyclyl is substituted with one or more —NR⁵SO₂R⁶ substituents.

In some embodiments, R¹ is a 6-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶. In some embodiments, R¹ is a 5-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶.

In some embodiments, the 3-10-membered heterocyclyl is substituted with one or more halogen substituents. In some embodiments, the 3-10-membered heterocyclyl is substituted with one or more C₁₋₆alkyl substituents. In some embodiments, the 3-10-membered heterocyclyl is substituted with one or more —O—C₁₋₆alkyl substituents. In some embodiments, the 3-10-membered heterocyclyl is substituted with one or more C₁₋₆haloalkyl substituents. In some embodiments, the 3-10-membered heterocyclyl is substituted with one or more —O—C₁₋₆haloalkyl substituents. In some embodiments, the 3-10-membered heterocyclyl is substituted with one or more C₂₋₆alkenyl substituents. In some embodiments, the 3-10-membered heterocyclyl is substituted with one or more C₂₋₆alkynyl substituents. In some embodiments, the 3-10-membered heterocyclyl is substituted with one or more —CN substituents. In some embodiments, the 3-10-membered heterocyclyl is substituted with one or more —CF₃ substituents. In some embodiments, the 3-10-membered heterocyclyl is substituted with one or more —OR⁵ substituents. In some embodiments, the 3-10-membered heterocyclyl is substituted with one or more —SR⁵ substituents. In some embodiments, the 3-10-membered heterocyclyl is substituted with one or more —COR⁵ substituents. In some embodiments, the 3-10-membered heterocyclyl is substituted with one or more halogen substituents. In some embodiments, the 3-10-membered heterocyclyl is substituted with one or more —CO₂R⁵ substituents. In some embodiments, the 3-10-membered heterocyclyl is substituted with one or more —CONR⁵R⁶ substituents. In some embodiments, the 3-10-membered heterocyclyl is substituted with one or more —NR⁵COR⁶ substituents. In some embodiments, the 3-10-membered heterocyclyl is substituted with one or more —SO₂R⁵ substituents. In some embodiments, the 3-10-membered heterocyclyl is substituted with one or more —SO₂NR⁵R⁶ substituents. In some embodiments, the 3-10-membered heterocyclyl is substituted with one or more —NR⁵SO₂R⁶ substituents.

In some embodiments, R² is a 6-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶. In some embodiments, R² is a 5-membered carbocyclyl and is substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR⁵, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶.

If present, each R³ and R⁴ are independently selected from the group consisting of hydrogen, C₁₋₆alkyl, 3-7-membered carbocyclyl and 3-7-membered heterocyclyl.

In some embodiments, R³ is hydrogen. In some embodiments, R³ is C₁₋₆alkyl. In some embodiments, R³ is 3-7-membered carbocyclyl. In some embodiments, R³ is 3-7-membered carbocyclyl. In some embodiments, R³ is a 5-membered carbocyclyl. In some embodiments R³ is a 6-membered carbocyclyl.

In some embodiments, R⁴ is hydrogen. In some embodiments, R⁴ is C₁₋₆alkyl. In some embodiments, R⁴ is 3-7-membered carbocyclyl. In some embodiments, R⁴ is 3-7-membered carbocyclyl. In some embodiments, R⁴ is a 5-membered carbocyclyl. In some embodiments R⁴ is a 6-membered carbocyclyl.

If present, each R⁵ and R⁶ are independently selected from the group consisting of hydrogen and C₁₋₆alkyl.

In some embodiments, R⁵ is hydrogen. In some embodiments, R⁵ is C₁₋₆alkyl.

In some embodiments, R⁶ is hydrogen. In some embodiments, R⁶ is C₁₋₆alkyl.

In some embodiments, R¹ and R² are each independently selected from the group consisting of hydrogen, halogen, 3-10-membered carbocyclyl, 3-10-membered heterocyclyl, —OH, —CN, and —NH₂. If present, each 3-10-membered carbocyclyl and 3-10-membered heterocyclyl may be further substituted with one or more substituents selected from the group consisting of hydrogen, halogen, —OH, —CN, —CF₃, —NH₂, and C₁₋₆alkyl.

In some embodiments, R¹ and R² are each independently selected from the group consisting of hydrogen, halogen, 6-membered carbocyclyl, 6-membered heterocyclyl, —OH, —CN, and —NH₂. If present, each 6-membered carbocyclyl and 6-membered heterocyclyl may be further substituted with one or more substituents selected from the group consisting of hydrogen, halogen, —OH, —CN, —CF₃, —NH₂, and C₁₋₆alkyl.

In some embodiments, R¹ is selected from the group consisting of hydrogen, halogen, —OH, —CN, —CF₃, —NH₂, and C₁₋₆alkyl, and R² is independently selected from the group consisting of:

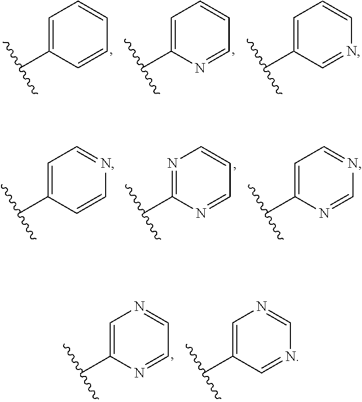

and

In some embodiments, R¹ is hydrogen and R² is independently selected from the group consisting of:

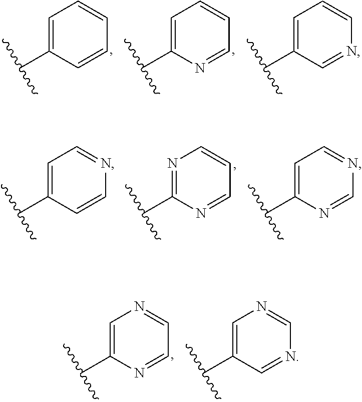

and

In some embodiments, R¹ is halogen and R² is independently selected from the group consisting of:

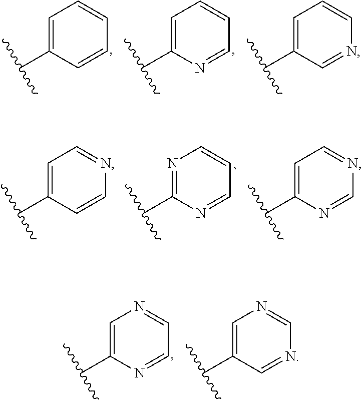

and

In some embodiments, R¹ is —OH and R² is independently selected from the group consisting of:

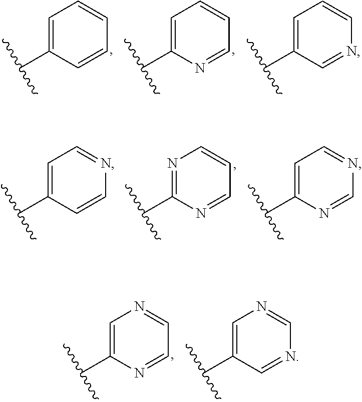

and

In some embodiments, R¹ is —CN and R² is independently selected from the group consisting of:

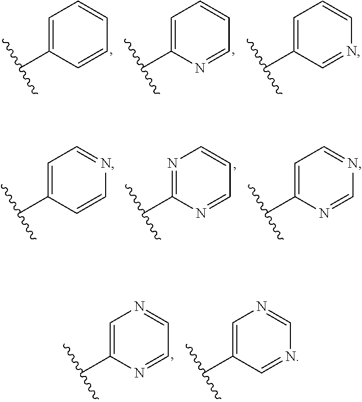

and

In some embodiments, R¹ is —CF₃ and R² is independently selected from the group consisting of:

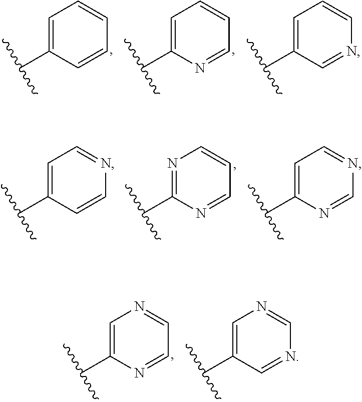

and

In some embodiments, R¹ is —NH₂ and R² is independently selected from the group consisting of:

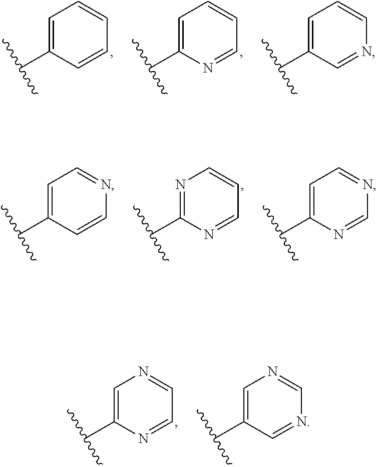

and

In some embodiments, R¹ is C₁₋₆alkyl and R² is independently selected from the group consisting of:

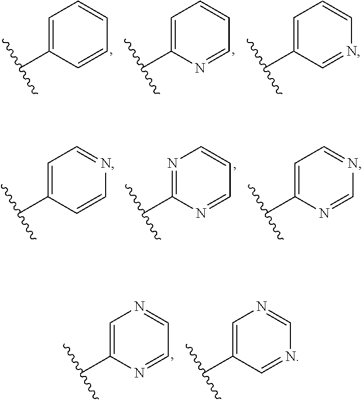

and

In some embodiments. R¹ is chlorine and R² is independently selected from the group consisting of:

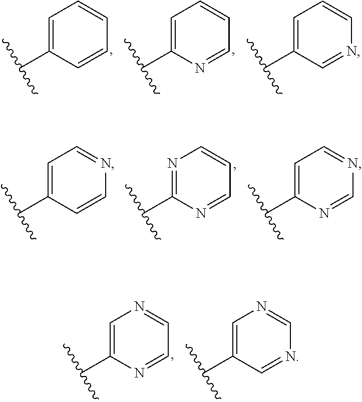

and

In some embodiments, R¹ is bromine and R² is independently selected from the

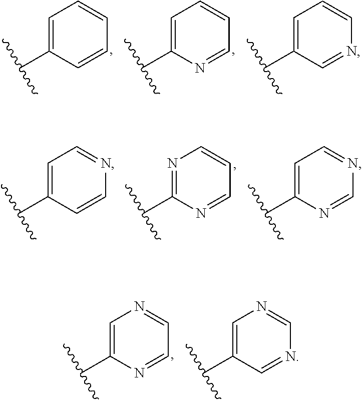

and

In some embodiments, R¹ is fluorine and R² is independently selected from the group consisting of:

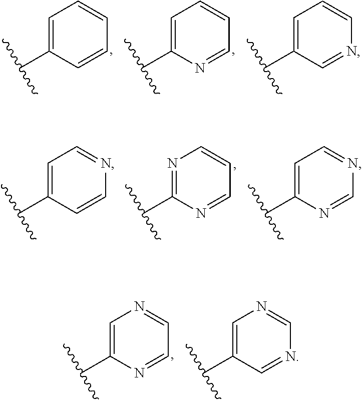

In some embodiments R¹ is iodine and R² is independently selected from the
and

In some embodiments, R¹ is iodine and R² is independently selected from the group consisting of:

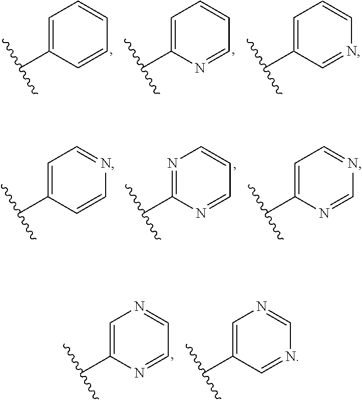

and

In some embodiments, R² is selected from the group consisting of hydrogen, halogen, —OH, —CN, —CF₃, —NH₂, and C₁₋₆alkyl, and R¹ is independently selected from the group consisting of:

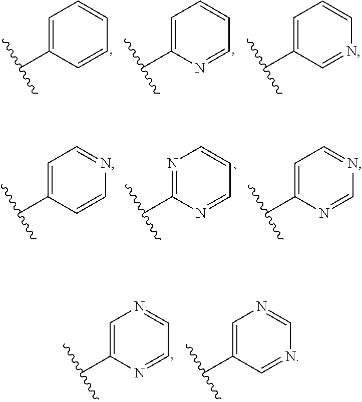

and

In some embodiments, R² is hydrogen and R¹ is independently selected from the group consisting of:

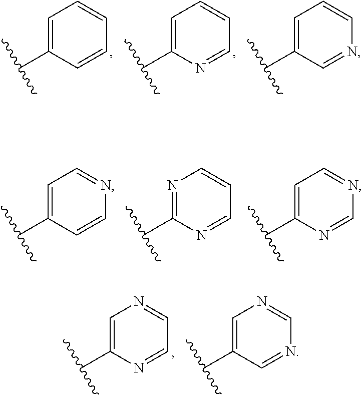

and

In some embodiments, R² is halogen and R¹ is independently selected from the group consisting of:

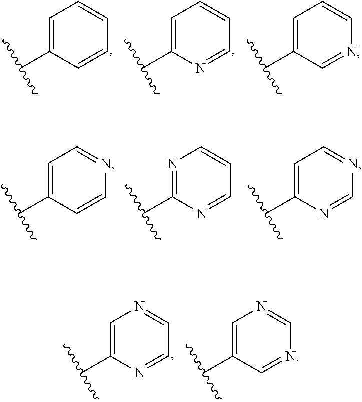

and

In some embodiments, R² is —OH and R¹ is independently selected from the group consisting of:

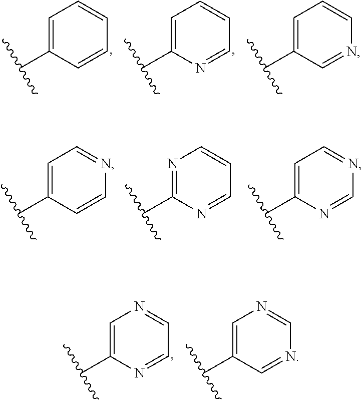

and

In some embodiments, R² is —CN and R¹ is independently selected from the group consisting of:

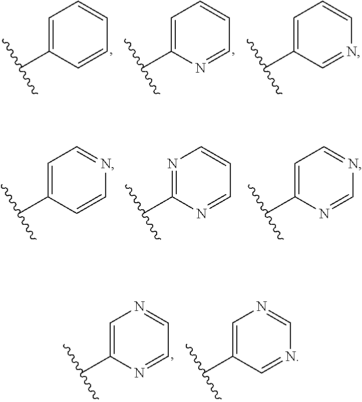

and

In some embodiments, R² is —CF₃ and R¹ is independently selected from the group consisting of:

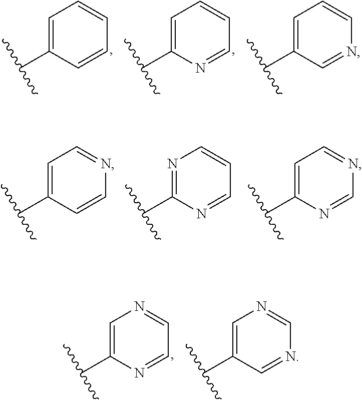

and

In some embodiments, R² is —NH₂ and R¹ is independently selected from the group consisting of:

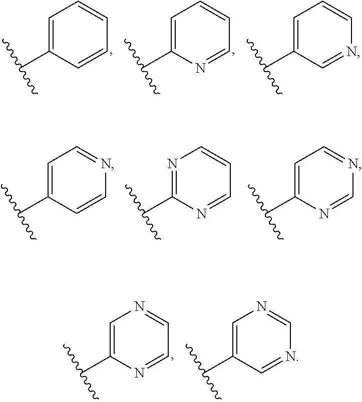

and

In some embodiments, R² is C₁₋₆alkyl and R¹ is independently selected from the group consisting of:

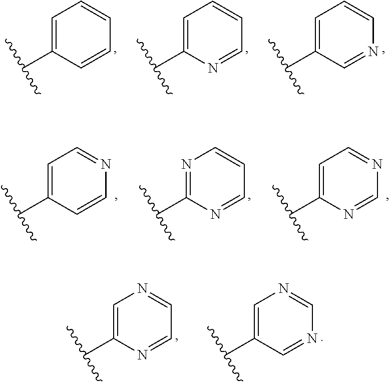

and

In some embodiments, R² is chlorine and R¹ is independently selected from the group consisting of:

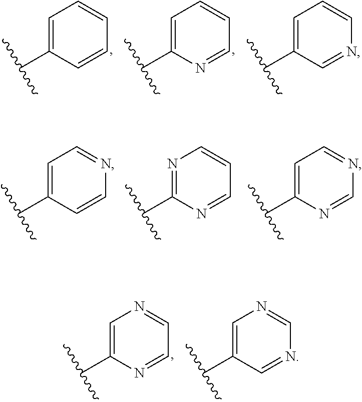

and

In some embodiments. R² is bromine and R¹ is independently selected from the group consisting of:

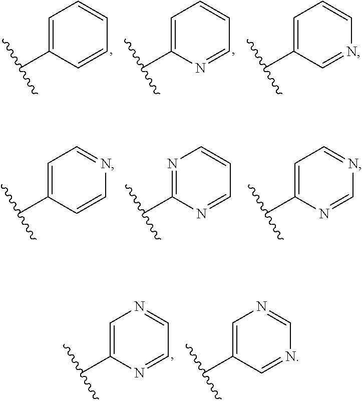

and

In some embodiments, R² is fluorine and R¹ is independently selected from the group consisting of:

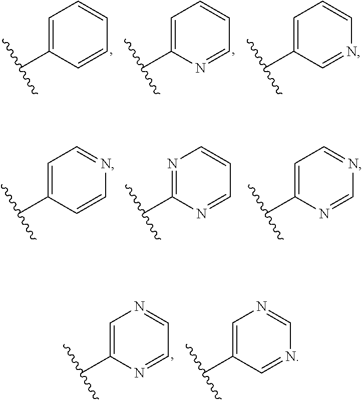

and

In some embodiments, R² is iodine and R¹ is independently selected from the group consisting of:

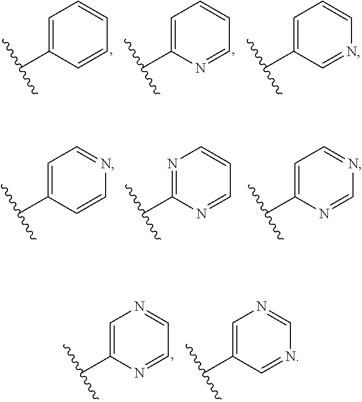

and

In some embodiments, R¹ and R² are each independently selected from the group consisting of:

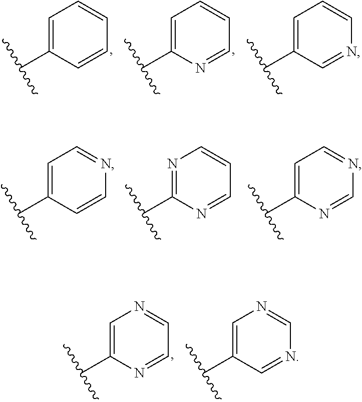

and

In some embodiments, the compound of Formula I is selected from the group consisting of:

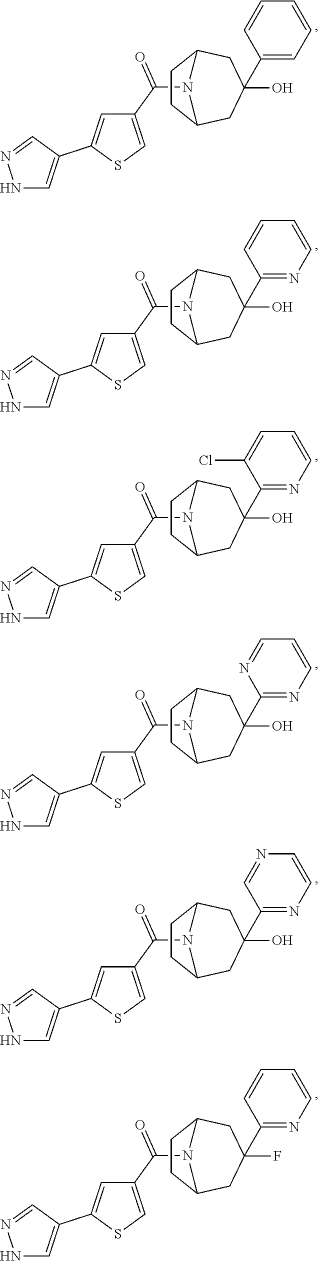 [SMILES: O=C(c1csc(-c2cn[nH]c2)c1)N1C2CCC1CC(O)(c1ccccc1)C2 ; O=C(c1csc(-c2cn[nH]c2)c1)N1C2CCC1CC(O)(c1ccccn1)C2 ; O=C(c1csc(-c2cn[nH]c2)c1)N1C2CCC1CC(O)(c1ncccc1Cl)C2 ; O=C(c1csc(-c2cn[nH]c2)c1)N1C2CCC1CC(O)(c1ncccn1)C2 ; O=C(c1csc(-c2cn[nH]c2)c1)N1C2CCC1CC(O)(c1cnccn1)C2 ; O=C(c1csc(-c2cn[nH]c2)c1)N1C2CCC1CC(F)(c1ccccn1)C2]

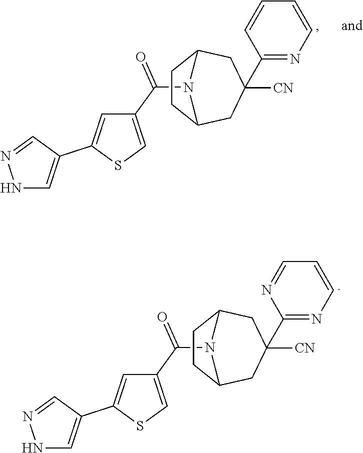 [SMILES: N#CC1(c2ccccn2)CC2CCC(C1)N2C(=O)c1csc(-c2cn[nH]c2)c1 ; N#CC1(c2ncccn2)CC2CCC(C1)N2C(=O)c1csc(-c2cn[nH]c2)c1]

In some embodiments, the compound of Formula I is:

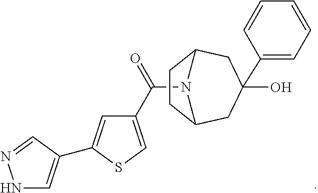 [SMILES: O=C(c1csc(-c2cn[nH]c2)c1)N1C2CCC1CC(O)(c1ccccc1)C2]

In some embodiments, the compound of Formula I is:

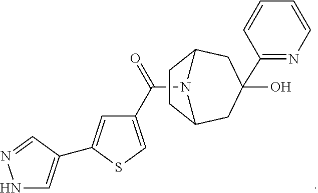 [SMILES: O=C(c1csc(-c2cn[nH]c2)c1)N1C2CCC1CC(O)(c1ccccn1)C2]

In some embodiments, the compound of Formula I is:

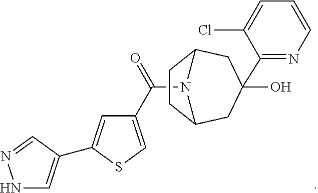 [SMILES: O=C(c1csc(-c2cn[nH]c2)c1)N1C2CCC1CC(O)(c1ncccc1Cl)C2]

In some embodiments, the compound of Formula I is:

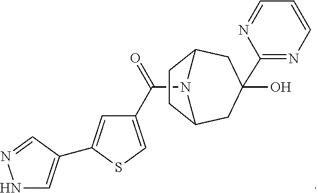 [SMILES: O=C(c1csc(-c2cn[nH]c2)c1)N1C2CCC1CC(O)(c1ncccn1)C2]

In some embodiments, the compound of Formula I is:

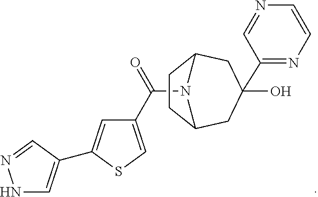 [SMILES: O=C(c1csc(-c2cn[nH]c2)c1)N1C2CCC1CC(O)(c1cnccn1)C2]

In some embodiments, the compound of Formula I is:

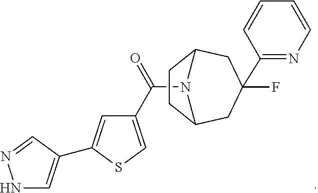 [SMILES: O=C(c1csc(-c2cn[nH]c2)c1)N1C2CCC1CC(F)(c1ccccn1)C2]

In some embodiments, the compound of Formula I is:

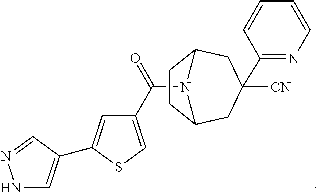 [SMILES: N#CC1(c2ccccn2)CC2CCC(C1)N2C(=O)c1csc(-c2cn[nH]c2)c1]

In some embodiments, the compound of Formula I is:

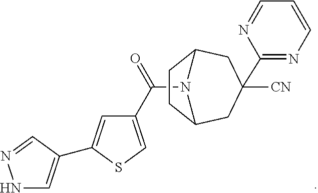 [SMILES: N#CC1(c2ncccn2)CC2CCC(C1)N2C(=O)c1csc(-c2cn[nH]c2)c1]

As would be understood by the person skilled in the art, the compound of Formula I includes any stereoisomers of the depicted structure. That is, the compound of Formula I includes a racemic mixture.

As used herein, a compound of Formula Ia has the following chemical structure:

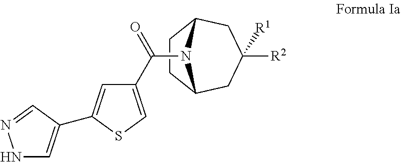

The definitions of R¹ and R² are the same as those provided for a compound of Formula I. That is, the difference between a compound of Formula I and a compound of Formula Ia is that the stereochemistry in a compound of Formula Ia has been resolved.

In some embodiments, the compound of Formula I, or a pharmaceutically acceptable salt, solvate or prodrug thereof, has a particular stereochemistry as depicted in the following chemical structure:

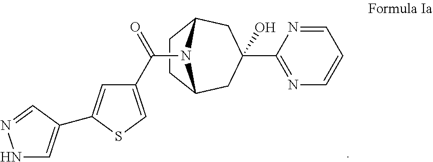 [SMILES: O=C(c1csc(-c2cn[nH]c2)c1)N1[C@@H]2CC[C@H]1C[C@](O)(c1ncccn1)C2]

The compound of Formula Ia, when having such particular stereochemistry, is also referred to as “UE2343” or “Xanamem”, and has CAS No.: 1346013-80-6. The chemical name (i.e., IUPAC name) of Formula Ia is (5-(1H-Pyrazol-4-yl)thiophen-3-yl)(3-hydroxy-3-(pyrimidin-2-yl)-8-azabicyclo[3.2.1]octan-8-yl)methanone.

A compound of Formula I, or a pharmaceutically acceptable salt, solvate or prodrug thereof, may be prepared by any suitable method as would be understood by the person skilled in the art. A compound of Formula I, or a pharmaceutically acceptable salt, solvate or prodrug thereof, may be prepared in accordance with the procedure described in WO2011135276, which is herein incorporated by reference thereto.

### Salts

It may be convenient or desirable to prepare, purify and/or handle a corresponding salt of the compound, such as, for example, a pharmaceutically acceptable salt. As used herein, the term “pharmaceutically acceptable salt” refers to pharmaceutically acceptable organic or inorganic salts. Examples of pharmaceutically acceptable salts are discussed in Berge et al., 1977, “Pharmaceutically Acceptable Salts,” J. Pharm. Sci., vol. 66, p1-19.

For example, if the compound is anionic, or has a functional group that may be anionic (e.g., —COOH may be —COO—), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R²⁺, NHR₃⁺, NR₄⁺). Examples of suitable substituted ammonium ions include, but are not limited to, those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group that may be cationic (e.g., —NH₂ may be —NH₃⁺), then a salt may be formed with a suitable anion. Examples of suitable inorganic cations include, but are not limited to, those derived from the inorganic acids including hydrochloric, hydrobromic, hydroiodic, sulfuric, sulfurous, nitric, nitrous, phosphoric, and phosphorous. Examples of suitable organic anions include, but are not limited to, those derived from the organic acids including 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicyclic, stearic, succinic, sulfanilic, tartaric, toluenesulfonic, and valeric. Examples of suitable polymeric organic anions include, but are not limited to, those derived from polymeric acids including tannic acid and carboxymethyl cellulose.

A pharmaceutically acceptable salt may involve the inclusion of another molecule such as an acetate ion, a succinate ion or other counterion. The counterion may be any organic or inorganic moiety that stabilises the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non-pharmaceutically acceptable salts also fall within the scope of the present disclosure since these may be useful as intermediates in the preparation of pharmaceutically acceptable salts or may be useful during storage or transport. Unless otherwise specified herein, reference to a particular compound also includes salts thereof.

### Solvates and Hydrates

It may be convenient or desirable to prepare, purify and/or handle a corresponding solvate of the compound. Those skilled in the art of organic chemistry and/or medicinal chemistry will appreciate that many organic compounds can form complexes with solvents in which they are reacted or from which they are precipitated or crystallised. Such complexes are referred to as “solvates”, and as used herein, the term “solvate” refers to such a complex of solute (e.g., a compound, salt of a compound) and solvent. Examples of solvents that may form pharmaceutically acceptable solvates include, but are not limited to, isopropanol, ethanol, methanol, DMSO, ethylacetate, acetic acid, and ethanolamine. If the solvate is water, the solvate may be conventionally referred to as a “hydrate”. In some embodiments, the pharmaceutically acceptable solvate is a pharmaceutically acceptable hydrate. The hydrate may be, for example, a mono-hydrate, a di-hydrate, a tri-hydrate, etc. Unless otherwise specified herein, reference to a particular compound also includes solvates thereof.

### Prodrugs

It may be convenient or desirable to prepare, purify, and/or handle the compound in the form of a prodrug. The term “prodrug”, as used herein, pertains to compound which, when metabolised (e.g., in vivo), yields the desired active compound. Typically, the prodrug is inactive, or less active that the desired active compound, but may provide advantageous handling, administration, or metabolic properties.

Also, as would be understood by a person skilled in the art of organic chemistry and/or medicinal chemistry, some prodrugs are activated enzymatically to yield the active compound, or a compound which, upon further chemical reaction, yields the active compound. For example, the prodrug may be a sugar derivative or other glycoside conjugate, or may be an amino acid ester derivative.

### Methods and Uses

The present disclosure provides for a method of treating or preventing depression in subjects resistant to antidepressant treatments and/or treating or preventing depression associated cognitive impairment in subjects resistant to antidepressant treatments. The methods can comprise administering to the subject a therapeutically effective amount of a compound of Formula I, or a pharmaceutically acceptable salt, solvate or prodrug thereof. The present disclosure also provides for use of a compound of Formula I, or a pharmaceutically acceptable salt, solvate or prodrug thereof, for treating depression in subjects resistant to antidepressant treatments and/or treating depression associated cognitive impairment in subjects resistant to antidepressant treatments. A compound of Formula I, or a pharmaceutically acceptable salt, solvate or prodrug thereof, may be used in the manufacture of a medicament for treating depression in subjects resistant to antidepressant treatments and/or treating depression associated cognitive impairment in subjects resistant to antidepressant treatments.

It will be appreciated that the dosage regimens and compositions as described herein may apply to any of the embodiments or examples of the methods as described herein.

### Dosage Regimen

As used herein, “therapeutically effective amount” refers to a compound of Formula I, or a pharmaceutically acceptable salt, solvate or prodrug thereof, being administered in an amount sufficient to alleviate or prevent to some extent one or more of the symptoms of the disorder or condition being treated, typically without undue adverse side effects or to achieve a desired pharmacological effect or therapeutic improvement with a reduced side effect profile. The results can be the reduction and/or alleviation of the signs, symptoms, or causes of a disease or condition, or any other desired alteration of a biological system. In some embodiments, the term “therapeutically effective amount” refers to a compound of Formula I, or a pharmaceutically acceptable salt, solvate or prodrug thereof, being administered in an amount sufficient to result in a reduction of symptoms associated with antidepressant resistant depression and/or associated cognitive impairment. Therapeutically effective amounts may, for example, be determined by routine experimentation, including but not limited to a dose escalation clinical trial. The phrase “therapeutically effective amount” includes, for example, a prophylactically effective amount. In some embodiments, a prophylactically effective amount is an amount sufficient to prevent antidepressant resistant depression and/or associated cognitive impairment. It is understood that “an effective amount” or “a therapeutically effective amount” can vary from subject to subject, due to variation in metabolism of the compound and any of age, weight, general condition of the subject, the condition being treated, the severity of the condition being treated, and the judgment of the prescribing physician. An appropriate an effective amount” or “a therapeutically effective amount” in any individual case may be determined by one of ordinary skill in the art using routine experimentation.

The amount of a compound of Formula I, or a pharmaceutically acceptable salt, solvate or prodrug thereof, that will be effective in the treatment and/or prevention of a particular disorder or condition disclosed herein will depend on the nature of the disorder or condition, and can be determined by standard clinical techniques. In addition, in vitro or in vivo assays may optionally be employed to help identify optimal dosage ranges. Such techniques are known to the person skilled in the art.

The precise dose to be administered to the subject will also depend on the route of administration, and the seriousness of the disease or disorder, and should be decided according to the judgment of the practitioner and each subject's circumstances. For example, suitable dosage ranges for oral administration, are generally from about 0.001 milligram to 1000 milligrams of the compound of Formula I, or a pharmaceutically acceptable salt, solvate or prodrug thereof per kilogram body weight.

In some embodiments, the compound of Formula I, or a pharmaceutically acceptable salt, solvate or prodrug thereof, is administered in an amount so as to deliver a total daily dosage (in mg) of at least about 1, 5, 10, 15, 20, 25, 30, 35, 40, 45, 50, 60, 75, 100, 150, or 200. In some embodiments, the compound of Formula I, or a pharmaceutically acceptable salt, solvate or prodrug thereof, is administered in an amount so as to deliver a total daily dosage (in mg) of less than about 200, 150, 100, 75, 60, 50, 45, 40, 35, 30, 25, 20, 15, 10, 5, or 1. The total daily dosage may be provided in a range between at any two of these upper and/or lower amounts. For example, a total daily dosage may be provided in an amount of between about 1 and 100 mg, about 5 and 75 mg, about 10 and 50 mg, about 15 and 45 mg, or about 20 and 40 mg.

In some embodiments, a therapeutically effective amount of a compound of Formula I, or a pharmaceutically acceptable salt, solvate or prodrug thereof, is administered to the subject at a predetermined frequency. In some embodiments, a compound of Formula I, or a pharmaceutically acceptable salt, solvate or prodrug thereof, is administered to the subject according to a dosage regimen in which a compound of Formula I, or a pharmaceutically acceptable salt, solvate or prodrug thereof is administered once daily, twice daily, three times daily, or four times daily. In some embodiments, the a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered once daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered twice daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered three times daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered four times daily. In some embodiments, a compound of Formula I, or a pharmaceutically acceptable salt, solvate or prodrug thereof is administered to the subject according to a dosage regimen in which a compound of Formula I, or a pharmaceutically acceptable salt, solvate or prodrug thereof is administered multiple times daily. In some examples, a compound of Formula I, or a pharmaceutically acceptable salt, solvate or prodrug thereof is administered as a once daily dose of between about 10 mg and 30 mg, for example at about 20 mg. In other examples, a compound of Formula I, or a pharmaceutically acceptable salt, solvate or prodrug thereof is administered as a twice daily dose of between about 5 mg and 20 mg per dose, for example at about 10 mg per dose. In some embodiments, a compound of Formula I, or a pharmaceutically acceptable salt, solvate or prodrug thereof is administered as a three-times daily dose of between about 5 mg and 15 mg per dose, for example at about 10 mg per dose.

In some embodiments, a therapeutically effective amount of a compound of Formula I, or a pharmaceutically acceptable salt, solvate or prodrug thereof, is administered to the subject at a predetermined frequency and/or duration. For example, administration according to any embodiments (e.g. frequency) as described herein may be for a duration of about, or at least about, 1 day, 1 week, 2 weeks, 3 weeks, 1 month, 2 months, 3 months, 4 months, 5 months, 6 months, 12 months, 2 years, or 5 years. Administration of the therapeutically effective amount of a compound of Formula I, or a pharmaceutically acceptable salt, solvate or prodrug thereof, may be ongoing so long as a therapeutic effect is received by the subject.

As used herein, the term “administer” and “administering” are used to mean introducing the compound of Formula I, or a pharmaceutically acceptable salt, solvate or prodrug thereof, into a subject. When administration is for the purpose of treatment, the compound of Formula I, or a pharmaceutically acceptable salt, solvate or prodrug thereof, is provided at, or after the onset of, a symptom of cognitive decline. The therapeutic administration of this substance serves to attenuate any symptom, or prevent additional symptoms from arising. When administration is for the purposes of preventing or reducing the likelihood cognitive decline, the compound of Formula I, or a pharmaceutically acceptable salt, solvate or prodrug thereof, is provided in advance of any visible or detectable symptom. The prophylactic administration of the compound of Formula I, or a pharmaceutically acceptable salt, solvate or prodrug thereof, serves to attenuate subsequently arising symptoms or prevent or reduce the likelihood of the symptoms from arising altogether.

A compound of Formula I, or a pharmaceutically acceptable salt, solvate or prodrug thereof, may be administered by any suitable route. Examples include, but are not limited to, oral, topical, transdermal, intranasal, vaginal, rectal, intraarterial, intramuscular, intraosseous, intraperitoneal, epidural and intrathecal. In some embodiments, a compound of Formula I, or a pharmaceutically acceptable salt, solvate or prodrug thereof, is administered orally.

A compound of Formula I, or a pharmaceutically acceptable salt, solvate or prodrug thereof, may be administered to the subject with respect to the subject's fasted state, as would be understood by the person skilled in the art. For example, the subject may be administered a compound of Formula I, or a pharmaceutically acceptable salt, solvate or prodrug thereof before, with, or after a meal. In some embodiments, a compound of Formula I, or a pharmaceutically acceptable salt, solvate or prodrug thereof is administered to the subject before a meal (i.e., the subject being in a fasted state). In some embodiments, a compound of Formula I, or a pharmaceutically acceptable salt, solvate or prodrug thereof is administered to the subject with a meal. In some embodiments, a compound of Formula I, or a pharmaceutically acceptable salt, solvate or prodrug thereof is administered at a certain interval (i.e., 30 mins, 1 hour, 2 hours, 3 hours, etc.) following a meal.

### Compositions

Compositions suitable for use in the methods and uses described herein comprise a compound of Formula I, or a pharmaceutically acceptable salt, solvate or prodrug thereof. In some embodiments, a compound of Formula I, or a pharmaceutically acceptable salt, solvate or prodrug thereof, is presented as a composition. In some embodiments, a compound of Formula I, or a pharmaceutically acceptable salt, solvate or prodrug thereof, is presented as a pharmaceutical composition.

The present disclosure also provides pharmaceutical compositions that comprise a compound of Formula I, or a pharmaceutically acceptable salt, solvate or prodrug thereof, with one or more pharmaceutically acceptable carriers, and optionally any other therapeutic ingredients, stabilisers, or the like. The carrier(s) must be pharmaceutically acceptable in the sense of being compatible with the other ingredients of the formulation and not unduly deleterious to the recipient thereof. Generally, suitable pharmaceutically acceptable carriers are known in the art and are selected based on the end use application. The pharmaceutically acceptable carrier may act as a diluent, dispersant or carrier for the active agents and other optional components of the composition. The pharmaceutically acceptable carrier may also contain materials commonly used in pharmaceutically products and can be in a wide variety of forms. For example, the carrier may be water, liquid or solid emollients, silicone oils, emulsifiers, surfactants, solvents, humectants, thickeners, powders, propellants and the like.

In some embodiments, the composition is a pharmaceutical composition, and wherein the composition comprises a pharmaceutically acceptable excipient.

The composition may for example contain a solvent, such as water (e.g. water for injection) or a pharmaceutically acceptable organic solvent.

The compositions may further include diluents, buffers, citrate, trehalose, binders, disintegrants, thickeners, lubricants, preservatives (including antioxidants), inorganic salts (e.g., sodium chloride), antimicrobial agents (e.g., benzalkonium chloride), sweeteners, antistatic agents, sorbitan esters, lipids (e.g., phospholipids such as lecithin and other phosphatidylcholines, phosphatidylethanolamines, fatty acids and fatty esters, steroids (e.g., cholesterol)), and chelating agents (e.g., EDTA, zinc and other such suitable cations).

The compositions of the present disclosure may also include polymeric excipients/additives or carriers, e.g., polyvinylpyrrolidones, derivatised celluloses such as hydroxymethylcellulose, hydroxyethylcellulose, and hydroxypropylmethylcellulose, Ficolls (a polymeric sugar), hydroxyethylstarch (HES), dextrates (e.g., cyclodextrins, such as 2-hydroxypropyl-β-cyclodextrin and sulfobutylether-β-cyclodextrin), polyethylene glycols, and pectin.

Other pharmaceutical carriers, excipients, optional ingredients and/or additives suitable for use in the compositions according to the present disclosure are listed in “Remington: The Science & Practice of Pharmacy”, 19.sup.th ed., Williams & Williams, (1995), and in the “Physician's Desk Reference”, 52.sup.nd ed., Medical Economics, Montvale, N.J. (1998), and in “Handbook of Pharmaceutical Excipients”, Third Ed., Ed. A. H. Kibbe, Pharmaceutical Press, 2000.

A compound of Formula I, or a pharmaceutically acceptable salt, solvate or prodrug thereof, of the present disclosure may be formulated in compositions including those suitable for inhalation to the lung, by aerosol, parenteral (including intraperitoneal, intravenous, subcutaneous, or intramuscular injection) or oral administration.

The compositions may conveniently be presented in unit dosage form and may be prepared by any of the methods well known in the art of pharmacy. All methods include the step of bringing a compound of Formula I, or a pharmaceutically acceptable salt, solvate or prodrug thereof, into association with a carrier that constitutes one or more accessory ingredients.

In general, the compositions are prepared by bringing a compound of Formula I, or a pharmaceutically acceptable salt, solvate or prodrug thereof into association with a liquid carrier to form a solution or a suspension, or alternatively, by bringing a compound of Formula I, or a pharmaceutically acceptable salt, solvate or prodrug thereof into association with formulation components suitable for forming a solid, optionally a particulate product, and then, if warranted, shaping the product into a desired delivery form.

In some embodiments, the composition is formulated for oral delivery. Compositions for oral delivery may, for example, be in the form of tablets, lozenges, aqueous or oily suspensions, granules, powders, emulsions, capsules, syrups, or elixirs. Orally administered compositions may contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in a tablet or pill form, the compositions may be coated to delay disintegration and absorption in the gastrointestinal tract thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade. The oral compositions described herein may contain from about 1% to about 95% of a compound of Formula I, or a pharmaceutically acceptable salt, solvate or prodrug thereof by weight, and the oral compositions may be dosed 1, 2, 3, 4, 5 or more times daily. The oral compositions described herein may contain a compound of Formula I, or a pharmaceutically acceptable salt, solvate or prodrug thereof by weight % in at least about 1, 5, 10, 15, 20, 25, 30, 35, 40, 45, 50, 55, 60, 65, 70, 75, 80, 85, or 90. The oral compositions described herein may contain a compound of Formula I, or a pharmaceutically acceptable salt, solvate or prodrug thereof by weight % in less than about 95, 90, 85, 80, 75, 70, 65, 60, 55, 50, 45, 40, 35, 30, 25, 20, 15, 10, or 5. The oral compositions described herein may contain a compound of Formula I, or a pharmaceutically acceptable salt, solvate or prodrug thereof by weight % in a range provided by any two of these upper and/or lower values, for example between about 5 and 20 wt %.

In some embodiments, the composition is formulated for parenteral delivery. For example, in one embodiment, the composition may be a sterile, lyophilized, crystalized or amorphous composition that is suitable for reconstitution in an aqueous vehicle prior to injection.

In one embodiment, a composition suitable for parenteral administration conveniently comprises a sterile aqueous preparation of a compound of Formula I, or a pharmaceutically acceptable salt, solvate or prodrug thereof, which may for example be formulated to be isotonic with the blood of the recipient.

Pharmaceutical compositions are also provided which are suitable for administration as an aerosol, by inhalation. These formulations comprise a solution or suspension of a compound of Formula I, or a pharmaceutically acceptable salt, solvate or prodrug thereof. The desired formulation may be placed in a small chamber and nebulized. Nebulization may be accomplished by compressed air or by ultrasonic energy to form a plurality of liquid droplets or solid particles comprising a compound of Formula I, or a pharmaceutically acceptable salt, solvate or prodrug thereof.

As discussed below, a compound of Formula I, or a pharmaceutically acceptable salt, solvate or prodrug thereof of the present disclosure may for example be administered in combination with one or more additional pharmaceutically active agents. Thus, in some embodiments, the composition comprises a compound of Formula I, or a pharmaceutically acceptable salt, solvate or prodrug thereof as defined herein, or a pharmaceutically acceptable salt thereof, one or more pharmaceutically acceptable carriers, and one or more additional pharmaceutically active agents.

Generally, the composition comprises a compound of Formula I, or a pharmaceutically acceptable salt, solvate or prodrug thereof in an amount that is therapeutically effective amount. In some embodiments, the therapeutically effective amount is provided by a single dose. In some embodiments, the therapeutically effective amount is provided by one or more doses administered as part of a course of treatment, for example, 1, 2, 3, 4, 5, 6, 7, 8, 9, 10, 11, 12, 13, 14, 15, 16, 17, 18, 19, 20, 21, 22, 23, 24, 25, 26, 27 or greater than 27 doses.

The person skilled in the art would understand that the amount of a compound of Formula I, or a pharmaceutically acceptable salt, solvate or prodrug thereof present in the composition will vary depending on the other ingredients present in the composition, the desired effect and the like. In some embodiments, the composition comprises a compound of Formula I, or a pharmaceutically acceptable salt, solvate or prodrug thereof in a concentration between about 0.001 to 1000 mg/mL, 0.01 to 500 mg/mL, 0.1 to 50 mg/mL. In some embodiments, the composition comprises a compound of Formula I, or a pharmaceutically acceptable salt, solvate or prodrug thereof in a concentration between about 1 to 99 wt %, 1 to 90 wt %, 1 to 85 wt %, 1 to 80 wt %, 1 to 75 wt %, 1 to 70 wt %, 1 to 65 wt %, 1 to 60 wt %, 1 to 55 wt %, 1 to 50 wt %, 1 to 45 wt %, 1 to 40 wt %, 1 to 35 wt %, 1 to 30 wt %, 5 to 99 wt %, 10 to 99 wt %, 15 to 99 wt %, 20 to 99 wt %, 25 to 99 wt %, 30 to 99 wt %, 35 to 99 wt %, 40 to 99 wt %, 45 to 99 wt %, 50 to 99 wt %, 54 to 99 wt %, 60 to 99 wt %, 65 to 99 wt %, 70 to 99 wt %, 75 to 99 wt %, 80 to 99 wt %, 85 to 99 wt %, 90 to 99 wt %, 5 to 90 wt %, 20 to 80 wt %, 30 to 70 wt %, or 40 to 60 wt %.

In some embodiments, a compound of Formula I, or a pharmaceutically acceptable salt, solvate or prodrug thereof may be administered in combination with a further therapeutic agent. The further therapeutic agent may be any one or more antidepressants according to any one or more examples thereof as described herein. For example, a compound of Formula 1 and any one or more SSRI and/or SNRI antidepressants according to any one or more examples thereof as described herein. In some embodiments, the methods and uses described herein also relate to co-administering one or more substances in addition to a compound of Formula I, or a pharmaceutically acceptable salt, solvate or prodrug thereof to the subject. The term “co-administer” indicates that each of at least two compounds are administered during a time frame wherein the respective periods of biological activity or effects overlap. Thus, the term includes sequential as well as coextensive administration of compounds. Similar to administering compounds, co-administration of more than one substance can be for therapeutic and/or prophylactic purposes. If more than one substance or compound is co-administered, the routes of administration of the two or more substances need not be the same. The scope of the methods and uses described herein are not limited by the identity of the substance or substances which may be co-administered with a compound of Formula I, or a pharmaceutically acceptable salt, solvate or prodrug thereof. For example, compositions comprising a compound of Formula I, or a pharmaceutically acceptable salt, solvate or prodrug thereof may be co-administered with fluids or other substances that are capable of alleviating, attenuating, preventing or removing symptoms in a subject suffering from, exhibiting the symptoms of, or at risk of suffering from cognitive decline. Types of fluid that can be co-administered with a compound of Formula I, or a pharmaceutically acceptable salt, solvate or prodrug thereof should be specific to the circumstances surrounding the particular subject that is suffering from, exhibiting the symptoms of, or at risk of suffering from a bacterial infection. For example, fluids that may be co-administered with a compound of Formula I, or a pharmaceutically acceptable salt, solvate or prodrug thereof include but are not limited to, electrolytes and/or water, salt solutions, such as sodium chloride and sodium bicarbonate, as well as whole blood, plasma, serum, serum albumin and colloid solutions.

The composition may be a food or beverage, or provided into a food or beverage. The composition may be a veterinary product, such as a dog or cat food product. The composition may be a feed additive, a supplement, or a medical food. In some embodiments, a compound of Formula I, or a pharmaceutically acceptable salt, solvate or prodrug thereof, may be incorporated into food products and beverages. The compound of Formula I, or pharmaceutically acceptable salt, solvate or prodrug thereof, may be impregnated, mixed, emulsified, sprayed or coated onto carriers such as cellulose, methylcellulose, dextrose, cyclodextrose, cyclodextrin, maltitol, and fibre. Delivery may also be enhanced with a range of surfactants, lipids, complexes, solvents and co-solvent pharmaceutical delivery systems known in the pharmaceutical art to improve bioavailability, absorption and efficacy. As used herein, the term “food” or “food product” includes any edible product for human or non-human consumption, such as but not limited to supplements, snacks (sweet and savory), cocoa-containing foods, flavours, beverages, dietary supplements and formulations including supplements used in animal health and nutrition. Additional ingredients desired in the resulting food product may be added at any point in the process.

The present disclosure includes food products comprising one or more compounds of Formula I alone as the active ingredient or in combination with other active ingredients.

In some embodiments, there is provided a composition comprising a compound of Formula I, or a pharmaceutically acceptable salt, solvate or prodrug thereof, and any one or more antidepressants according to any one or more examples thereof as described herein. For example, a composition comprising a compound of Formula 1 and any one or more SSRI and/or SNRI antidepressants according to any one or more examples thereof as described herein.

It will be appreciated that administration of the compound may be provided in various forms depending on the application and subject.

### EXAMPLES

### General: Materials and Methods

Formulations comprising a compound of Formula Ia were tested on human subjects to evaluate the use in preventing and/or treating antidepressant resistant depression and associate cognitive impairment.

The compound of Formula Ia, as described herein, was synthesised and characterised according to the procedure described in WO2011135276, which is herein incorporated by reference thereto. The compound of Formula Ia is also known as UE2343 or (5-(1H-pyrazol-4-yl)thiophen-3-yl)(3-hydroxy-3-(pyrimidin-2-yl)-8-azabicyclo[3.2.1]octan-8-yl)methanone. Xanamem™ contains the compound of Formula Ia as an active ingredient.

### Example 1

XanaMDD: A Double-Blind, Placebo-Controlled, Parallel-Groups Study to Assess the Efficacy on Cognition and Depression and Safety of Xanamem™ 10 mg daily in patients with treated Major Depressive Disorder with ongoing Depression and Cognitive Impairment.
Study Centre: Multicenter, up to 10 sites in Australia.
Study Design: This is a randomized, placebo-controlled, parallel-groups, double-blind trial in individuals with a confirmed diagnosis of a current episode of MDD who continue to experience cognitive difficulty and depressive symptoms.

Individuals aged ≥18 and ≤70 years, diagnosed according to DSM V criteria with a current MDD episode using the Mini International Neuropsychiatric Interview (Lecrubier et al., 1997) at screening, performed by a psychiatrist, will be eligible for inclusion in the trial. Participants have been treated with an SSRI, SNRI or atypical antidepressant for at least 6 weeks and have persistent symptoms of depression (HAM-D>17) and poor cognition as determined by a patient self-report. Trial participants will also be required to exhibit a 0.5 SD impairment on the WAIS Coding Test relative to age, education and gender relevant norms to demonstrate cognitive impairment. Participants will be prescreened to evaluate likely eligibility. Following consent, a psychiatric interview will be conducted, and the WAIS Coding Test performed to confirm psychiatric and cognitive inclusion/exclusion criteria. Screening blood samples will also be collected.

Participants will participate in the trial for up to 14 weeks. The trial consists of a screening period (maximum 4 weeks), a treatment period of 6 weeks and follow-up period (4 weeks). Participants will be randomly assigned 1:1 to Xanamem 10 mg or matching placebo daily. Approximately 120 participants will be enrolled. Those participants who meet the eligibility criteria with screening bloods collected ≤14 days before their scheduled Day 1 will proceed directly to Day 1, Baseline Treatment Visit. Participants whose Screening Visit occurred >14 days and in whom eligibility has not yet been confirmed will be required to attend the study site between Day −3 to Day −1 for Screening Visit 2 to collect additional baseline clinical laboratory assessments.

On Day 1 prior to dosing participants will have inclusion/exclusion criteria confirmed, including confirmation of on-going depressive symptoms (HAM-D) and cognitive difficulty (WAIS Coding). Baseline assessments will be performed, including two Cogstate Neuropsychological Test Battery (NTB) assessments at least 1 hour apart. The NTB is comprised of digital and paper-based measures of attention, executive function and episodic memory domains.

They will take investigational product at the site, with or after food, and in accordance with the randomization schedule.

At all follow up visits, participants should take the investigational product at home with or after food and have the time of their dose recorded in the drug intake diary. If participants forget to take their investigational product prior to the visit they may take it at the study site (or at home later if they did not bring their medication to clinic). A follow-up visit will be performed 4 weeks after the last dose of investigational product, for final safety and efficacy durability assessments. Further details of the study are provided below:

Objectives and Endpoints:

| Study Objectives | Study Endpoints |
|---|---|
| Efficacy Objectives | Primary Efficacy Endpoints |
| To determine the effects of | Change from baseline in the |
| Xanamem on Attention including | Attention Composite of the |
| Working Memory | Cogstate NTB to the end of |
| | treatment |
| To determine the effects of | Change from baseline on MADRS |
| Xanamem on depressive symptoms | to the end of treatment |
| | Secondary Efficacy Endpoints |
| To determine the effects of | Change from baseline in the |
| Xanamem on various aspects of | Executive Function composite |
| cognition | Change from baseline in the |
| | Episodic Memory Composite |
| | Change from baseline in individual |
| | tests of the Cogstate NTB and |
| | other neuropsychological tests |
| | (Hopkins Verbal Learning Test, |
| | Controlled Word Association Test, |
| | Trail Making A and B Tests) |
| To determine the durability of | Durability of cognitive and anti- |
| antidepressant response to | depressive effects after 4 weeks of |
| Xanamem | follow up |
| Safety Objectives | Endpoints |
| To evaluate the short-term safety | Incidence and severity of adverse |
| and tolerability of Xanamem | events; |
| | Incidence of serious adverse events |
| | and suspected unexpected serious |
| | adverse reactions; |
| | Clinically significant changes from |
| | baseline in: |
| | Clinical safety laboratory |
| | evaluations; |
| | Electrocardiograms; |
| | Vital signs; |
| | Physical examinations |
| | Columbia-Suicide Severity |
| | Rating Scale |
| PK Objectives | Endpoints |
| Confirm Xanamem exposure and | Sparse pharmacokinetic sampling |
| contribute to a population | for use in population PK analysis |
| pharmacokinetic model | |

| Safety Review | A SRC comprising lead investigator(s), a Sponsor Medical Monitor and an | |
|---|---|---|
| Committee | Independent Medical Monitor, who will act as Chairperson, will meet periodically to | |
| | review blinded safety data and overall trial conduct. The SRC or its Chairperson will | |
| | be able to request unblinding of individual participants if required for safety | |
| | interpretation. | |
| Investigational : | All Participants will receive a single dose of investigational product (10 mg | |
| Product | Xanamem or placebo) in the morning with or after food every day for 6 weeks. Both | |
| Administration | the site staff treating Participants and the Participants themselves will be blinded to | |
| | the treatments being administered. | |
| Inclusion | To be eligible for study entry Participants must satisfy all of the following criteria: | |
| Criteria: | 1. | Male or female aged 18 to 70, inclusive at the time of screening; |
| | 2. | Positive MDD primary diagnosis confirmed by Mini International |
| | | Neuropsychiatric Interview at screening; |
| | 3. | Persistent depressive symptoms as determined by a HAM-D >17 at |
| | | Screening and Baseline |
| | 4. | Self-reported subjective cognitive dysfunction such as difficulty |
| | | concentrating, slow thinking and difficulty in learning new things or |
| | | remembering things |
| | 5. | On a stable dose of a SSRI, SNRI or atypical antidepressant drug for at |
| | | least 6 weeks |
| | 6. | Exhibit a −0.5 SD impairment on the WAIS Coding Test relative to age, |
| | | education and gender relevant norms at Screening and Baseline. |
| | 7. | Body mass index 17.5 to 35 kg/m², inclusive at the time of screening; |
| | 8. | Must provide written informed consent to participate in the study and be |
| | | willing and able to participate for the site visits; |
| | 9. | Must be willing and able to comply with the requirements of the protocol |
| | | and must be available to complete the study; |
| | 10. | Female participants: Unless post-menopausal or male partners are known to |
| | | be infertile, female participants of childbearing potential must agree to use |
| | | an acceptable, highly reliable method of contraception during the study and |
| | | for 30 days after the last dose of study treatment. They must have a |
| | | negative serum pregnancy test at screening and a negative urine pregnancy |
| | | test at Day 1 and must not be breastfeeding, lactating or planning pregnancy |
| | | during the study period. WOCBP must agree to use an acceptable form of |
| | | contraception during the treatment period and for at least 30 days after the |
| | | last dose of study treatment. |
| | 11. | Male participants: Unless known to be infertile, a male subject with a |
| | | female partner of childbearing potential is only eligible to participate if he, |
| | | and his partner, agree to use an acceptable, highly reliable method of |
| | | contraception during the treatment period and for at least 90 days after the |
| | | last dose of study treatment and if he refrains from donating sperm during |
| | | this period. |
| | Participants will be excluded from the study if one or more of the following criteria | |
| | are applicable: | |
| | 1. | Active suicidal ideation (“yes” to any of questions 3, 4, 5 on C-SSRS |
| | | and/or per Investigator assessment). |
| | 2. | Other comorbid psychiatric disorder of clinical concern, as determined by |
| | | medical history and screening psychiatric assessment |
| | 3. | A history of severe depression treated with electroconvulsive therapy or |
| | | transcranial magnetic stimulation. |
| | 4. | Abnormalities in resting vital signs at screening (blood pressure >160/100, |
| | | heart rate >90, respiration rate >25 and clinically significant elevated oral |
| | | temperature), or any clinically significant abnormalities in vital signs |
| | | requiring intervention in the opinion of the Investigator. Up to 2 repeat |
| | | measurements according to investigator discretion are permitted to confirm |
| | | eligibility. |
| | 5. | Clinically significant abnormal hematology or biochemistry values, as |
| | | determined by the investigator at screening. The following values will have |
| | | an alert flag on the laboratory report and are specifically excluded: |

| | |
|---|---|
| i. | Hemoglobin ≤12 g/dL (≤120 g/L) for females and ≤13 g/dL |
| | (≤130 g/L) for males |
| ii. | Aspartate aminotransferase >1.5 x upper limit of normal (ULN) |
| iii. | Alanine aminotransferase >1.5 x ULN |
| iv. | Creatinine enzymatic eGFR <40 ml/min |

| | |
|---|---|
| 6. | Participants who have a history of liver disease requiring medical |
| | intervention, including fatty liver, or persistent liver function test |
| | elevations, unless due to Gilbert's syndrome; |
| 7. | Previous clinically significant systemic illness or infection, including test- |
| | positive COVID-19, within the past 4 weeks prior to screening, as |
| | determined by the investigator; |
| 8. | Clinical diagnosis of Type I or Type II diabetes requiring insulin; |
| 9. | History of clinically significant hypoglycemia in the past 5 years; |
| 10. | History of disease directly related to the hypothalamus, the pituitary and/or |
| | the adrenal glands which affects the hypothalamic-pituitary-adrenal axis |
| | function, e.g., Cushing's Disease, Adenoma, Conn-Syndrome, other |
| | malignancies; |
| 11. | Medically diagnosed peripheral neuropathy; |
| 12. | Clinically significant electrocardiogram abnormalities, including QTcF |
| | interval >450 msec, (male) and >470 msec (female), other than controlled |
| | atrial fibrillation (on rate control medication with heart rate ≤90 bpm at |
| | screening) at screening; |
| 13. | Smokers of >5 cigarettes a day or equivalent nicotine (includes vaping) / |
| | tobacco products |
| 14. | Smokers of ≤5 cigarettes a day or equivalent nicotine (includes vaping) / |
| | tobacco products who are unwilling to abstain from nicotine / tobacco |
| | products on the cognitive testing days. |
| 15. | Concomitant treatment with stimulant drugs (eg methylphenidate, |
| | (lis)dexamphetamine) for ADHD |
| 16. | Regular use of benzodiazepine or similar sedative drugs in the daytime. |
| | Nocturnal use of hypnotics, if stable, is allowed. |
| 17. | Chronic opioid therapy using an S8 drug. Intermittent use of codeine or |
| | short-acting tramadol is allowed provided not on the day of cognitive |
| | assessment. |
| 18. | Participation in another clinical study of a drug or device whereby the last |
| | investigational drug/device administration is within 30 days of screening, |
| | or 5 half-lives of the investigational drug, whichever is longer; |
| 19. | Known allergy to the study drug (Xanamem) or any of the excipients of the |
| | formulation (lactose, talc, magnesium stearate, croscarmellose sodium); |
| 20. | Study participants who in the opinion of the investigator exhibit physical, |
| | cognitive, or language impairments of such severity as to adversely affect |
| | the validity of the data derived from the neuropsychological tests); |
| 21. | Positive testing for human immunodeficiency virus, hepatitis B surface |
| | antigen, or hepatitis C antibodies at screening. Participants returning a |
| | positive result will be managed by the site in line with standard care; |
| 22. | Participants with a history of drug abuse or addiction in the past 5 years. |
| | Urinary drug testing results at screening not consistent with medical history |
| | is an exclusion; |
| 23. | Evidence or history of alcohol abuse (defined as greater than 21 standard |
| | units per week for males and greater than 14 standard units per week for |
| | females). |

| | |
|---|---|
| Sample Size: | The sample size for this study is 120 patients. |

### Formulations of Xanamem™ and Placebo Capsules

TABLE 1
Xanamem ™ 10 mg capsule formulation

| | | Formulation |
|---|---|---|
| Component | Weight per unit | percentage % w/w |
| UE2343 powder | 10 mg | 10.53 |
| Lactose | 170.5 mg | 84.47 |
| Croscarmellose Sodium | 7.6 mg | 4.00 |
| Talc | 0.95 mg | 0.50 |
| Magnesium Stearate | 0.95 mg | 0.50 |
| Total | 190 mg | 100% |

TABLE 2
Placebo capsule formulation

| | | Formulation |
|---|---|---|
| Component | Weight per unit | percentage % w/w |
| Lactose | 180.5 mg | 95 |
| Croscarmellose Sodium | 7.6 mg | 4.00 |
| Talc | 0.95 mg | 0.50 |
| Magnesium Stearate | 0.95 mg | 0.50 |
| Total | 190 mg | 100% |

The Cogstate Test Battery can include the following tests: detection test, identification test, one card learning test, one back working memory test, and the continuous paired associate learning test (CPAL; paired associate learning).

### Detection Test

The Cogstate Detection test is a measure of simple reaction time and has been shown to provide a valid assessment of psychomotor function. For this test, the subject pressed a “YES” response key as soon as they detected an event (a card turning face up presented in the centre of the computer screen). The software measured the response time to detect each event.

### Identification Test

The Cogstate Identification test is a measure of choice reaction time and has been shown to provide a valid assessment of visual attention. In this test an event (a card turning face up) occurred in the centre of the computer screen and the subject decided “YES” or “NO” as to whether this event met a predefined and unchanging criterion (i.e. is the color of the card red?). The software measured the speed and accuracy of each response.

### One Card Learning Test

The One Card Learning test is a continuous visual recognition learning test that assesses visual learning within a pattern separation model (Yassa et al., 2010). Theoretical models of the pattern separation model specify that information is organized in orthogonal and distinct non-overlapping representations so that new memories can be stored rapidly without interference. The One Card Learning test has been shown to be a valid test of learning and memory.

In this test, the participant attended to the card in the centre of the screen and responded to the question “have you seen this card before in this test?” If the answer was yes, participants were instructed to press the “YES” button, otherwise they were to press the “NO” button if the answer was no. Normal playing cards were displayed (without joker cards).

In this test, six cards were drawn at random from the deck and were repeated throughout the test. These four cards were interspersed with distractors (non-repeating cards). The test ended after 50 trials, without rescheduling for post-anticipatory correct trials. The primary performance measure for this test was the proportion of correct answers (accuracy), which was normalized using an arcsine square root transformation.

### One Back Working Memory Test

The Cogstate One Back memory test is a valid measure of working memory. On this test the subject was shown a single stimulus in the centre of the computer screen (a card turns face up). The subject decided “YES” or “NO” as to whether the current card matched the card that had been seen on the immediately previous trial. The software measured the speed and accuracy of each response.

### Continuous Paired Associate Learning Test (CPAL; Paired Associate Learning)

The Continuous Paired Associate Learning test is a measure of visual associate memory and uses a well-validated paired associate learning paradigm in which the subject must learn the locations of a number of amoeba-like shapes on the computer screen.

This test consisted of a single amoeboid shape displayed in the centre of the screen surrounded by a number of blue-filled circles. Beneath all but two of the blue spheres were amoeboid shapes, one of which matched the central display; the two remaining circles were distractors. In the exposure phase of the test all of the to-be-remembered pattern-location associations were presented on the computer screen simultaneously.

After a five second delay, a pattern was shown in the central location and this signals that the subject should touch the location in the periphery that contained the same pattern. This process continued until the participant had acknowledged all of the pattern-location associations. The learning phase began with the same test display presented during the exposure phase except that now all of the peripheral locations were shown as blue spheres. One of the patterns presented in the exposure phase was presented in the centre location. With the presentation of this pattern, the subject was required to select the peripheral location where an identical pattern was hidden beneath the blue sphere. This process continued until the correct location of each pattern was found.

Finding the correct location for all patterns in the set was defined as a learning trial. There were six learning trials. A single trial delayed recall condition was available for this test after a 10-30 minute delay. The software recorded each move as an error or as a correct move.

### Timing of Cogstate Test Battery

The timing of the Cogstate Test Battery is shown in Table 3.

TABLE 3
Trials and timing for each test in the cognitive battery

| | N Trials/ | Maximum | Average time |
|---|---|---|---|
| Test | Screens | Time Allowed | Required |
| Subject demographic screen | 1 | Unlimited | ~1 minute |
| Detection test instructions & demo | 1 | Unlimited | ~1 minute |
| Detection test | 35 | 5 minutes | ~2 minutes |
| Identification test instructions & demo | 1 | Unlimited | ~1 minute |
| Identification Test | 30 | 5 minutes | ~2 minutes |
| One Card Learning test instructions & demo | 1 | Unlimited | ~1 minute |
| One Card Learning Test | 80 | 5 minutes | ~4 minutes |
| One Back test instructions & demo | 1 | Unlimited | ~1 minute |
| One Back test | 31 | 5 minutes | ~3 minutes |
| Continuous Paired Associate Learning Test | 1 | Unlimited | ~1 minute |
| instructions & demo | | | |
| Continuous Paired Associate Learning Test | 7 | Unlimited | ~7 minutes |
| Continuous Paired Associate Learning Test - | 1 | 2 minutes | ~1 minute |
| Delayed Recall | | | |
| Estimated total time cognitive battery | | | ~25 minutes |

### Cognitive Outcome Measures

Although each of the cognitive tests to be used yields multiple outcome measures, research by Cogstate identified a set of measures that were optimal for the detection of cognitive change in clinical trials at both the group and individual level.

For each cognitive test, a single outcome measure was selected from each test in the battery to minimize experiment-wise error rates. Each outcome measure was selected because it was shown to be optimal for the detection of change because:
it was drawn from a data distribution that contained only a small probability of floor or ceiling effects and no restriction in the range of possible performance values; and
it was drawn from a distribution that is normal or which can be corrected to normal through the use of appropriate mathematical transformation (e.g., logarithmic base 10, or arcsine).

The outcome measures for the test used are summarized in Table 4.

TABLE 4
Recommended outcome measures for the cognitive battery

| | | | Range | |
|---|---|---|---|---|
| | | Unit of | of | |
| Test name | Cognitive domain | measurement | scores | Description |
| Detection Test | Psychomotor | Log₁₀ | 0-3.69* | Speed of performance; |
| | function | milliseconds | | mean of the log₁₀ |
| | | | | transformed reaction times |
| | | | | for correct responses |
| Identification | Visual attention | Log₁₀ | 0-3.69* | Speed of performance; |
| Test | | milliseconds | | mean of the log₁₀ |
| | | | | transformed reaction times |
| | | | | for correct responses |
| One Card | Visual learning | Arcsine | 0-1.57 | Accuracy of performance; |
| Learning Test | | proportion | | arcsine transformation of |
| | | correct | | the proportion of correct |
| | | | | responses |
| One Back Test | Working memory | Log₁₀ | 0-3.69 | Speed of performance; |
| | | milliseconds | | mean of the log₁₀ |
| | | | | transformed reaction times |
| | | | | for correct responses |
| Continuous | Hippocampal | Number of | 0-999 | Number of errors |
| Paired Associate | dependent learning | errors across all | | remembering the |
| Learning Test | and memory | learning trials | | associations |
| Continuous | Hippocampal | Number of | 0-999 | Number of errors |
| Paired Associate | dependent learning | errors across | | remembering the |
| Learning Test - | and memory | single delayed | | associations after a delay |
| Delayed Recall | | trial | | |

*Reaction times longer than 5 seconds (i.e. log10 [5000]) were excluded as reflecting responses that were abnormally slow. # for this test total n errors in a single administration was controlled so that patients who did not understand the test were not tested unnecessarily.

### Example 2

XanaCIDD: A Double-Blind, Randomized, Placebo-Controlled, Parallel-Group Trial to Assess the Safety, Tolerability, and Efficacy of Xanamem® in Adults with Cognitive Impairment due to Depressive Disorder.
Trial Centres: This clinical trial will be conducted at multiple sites in Australia and the United Kingdom.
Study Design: This is a Phase 2, randomized, placebo-controlled, parallel-group, double-blind, proof-of-concept trial in individuals with a confirmed diagnosis of MDD and who continue to experience cognitive impairment and depressive symptoms.

Individuals aged ≥18 and ≤75 years, diagnosed according to Diagnostic and Statistical Manual of Mental Disorders, Fifth Edition (DSM-5) criteria with a current MDD episode using the Mini-International Neuropsychiatric Interview (MINI) at Screening, performed by an accredited health professional, will be eligible for inclusion in the trial. Participants must be on a stable dose of a first- or second-line antidepressant that is approved for the treatment of depression (but not a tricyclic antidepressant, monoamine oxidase inhibitor, or vortioxetine) for at least 6 weeks or have a history of prior antidepressant use, have persistent symptoms of depression (Hamilton Depression Rating scale [HAM-D]≥17), and poor cognition as determined by an affirmative response from the participant to any aspect of a single-question test about perceived problems with their thinking (‘Questions About Cognition’). Trial participants will also be required to exhibit a 0.5 SD impairment on the Brief and Simple Index of Cognition (BASIC) Boxfiller subtest, relative to age and education relevant norms, to demonstrate cognitive impairment.

Following provision of informed consent, assessments will be conducted according to the Schedule of Assessments (Table 8). The MINI will be performed to confirm relevant inclusion/exclusion criteria. Participants will complete a familiarization session and practice administration for each of the cognitive efficacy assessments during the Screening period to ensure they understand each procedure and to reduce any potential learning effects during the treatment period.

Participants will take part in the trial for up to 14 weeks. The trial consists of a screening period (maximum 4 weeks), a treatment period of 6 weeks and a follow-up period (4 weeks). Participants will be randomly assigned 1:1 to receive 10 mg Xanamem or matching placebo daily. Approximately 160 participants will be enrolled.

Those participants who meet the eligibility criteria with screening bloods collected ≤14 days before their scheduled Day 1 visit will proceed directly to Day 1, Baseline Visit. Participants whose Screening Visit occurred >14 days prior to their scheduled Day 1 visit will be required to attend the trial site between Day −13 to Day −3 for Screening Visit 2 to collect additional Screening blood samples and for review of medical history.

During the Day 1 Baseline Visit, all Baseline assessments will be performed in accordance with the Schedule of Assessments (Table 8). The cognitive assessments comprise digital and paper-based measures of attention, executive function, and episodic memory domains and include selected tests from the Cogstate CTB.

On Day 1, participants will take investigational product (IP) at the site, with or without food, and in accordance with the randomization schedule. Both the site staff treating participants and the participants themselves will be blinded to the treatments being administered.

Three (3) follow-up visits will be conducted during the treatment period, at Weeks 2, 4, and 6 (EOT Visit) for safety and efficacy assessments. Some visits may be conducted remotely from trial sites. On the day of all follow-up visits, participants should take the IP at home with or without food in the morning and have the time of their dose recorded in their drug intake diary. If participants are attending a clinic for visits and they forget to take their IP prior to the visit they may take it at the trial site (or at home later if they did not bring their medication to the clinic).

A final Follow-up Visit at Week 10 (EOS Visit) will be performed 4 weeks after the last dose of IP for final safety and efficacy durability assessments. Further details of the study are provided below:

Objectives and Endpoints:

| Trial Objectives | Trial Endpoints |
|---|---|
| Efficacy Objectives | Primary Efficacy Endpoint |
| To determine the effects of | Change from Baseline to the end of treatment |
| Xanamem on attention, | (EOT) in an Attention Composite of a |
| including working memory | cognitive test battery (CTB) (Detection, |
| | Identification, and One Back tests) |
| | Secondary Efficacy Endpoints |
| To determine the effects of | Change from Baseline to EOT on the |
| Xanamem on depressive | Montgomery-Åsberg Depression Rating |
| symptoms | Scale (MADRS) |
| To determine the effects of | Change from Baseline to EOT in an |
| Xanamem on executive | Executive Function Composite (One Back |
| function | Test, Category Fluency Test [CFT], Letter |
| | Fluency Test [LFT], and International Digit |
| | Symbol Substitution Test - Symbols [IDSST-S]) |
| To determine the effects of | Change from Baseline to EOT in an Episodic |
| Xanamem on episodic | Memory Composite (One Card Learning and |
| memory | Hopkins Verbal Learning Test - Revised |
| | [HVLT-R], Immediate Recall only) |
| To determine the effects of | Change from Baseline to EOT in individual |
| Xanamem on individual | tests of the CTB including Cogstate tests and |
| cognitive tests that assess | other cognitive tests (HVLT-R, CFT, and |
| attention (including | LFT) |
| working memory), | |
| executive function, and | |
| episodic memory | |
| To determine the responder | Percentage of participants achieving a 50% |
| rate of Xanamem on | or greater reduction in MADRS score |
| depressive symptoms | Percentage of participants achieving a |
| | MADRS score of <10 points at Week 6 |
| | (EOT) |
| To determine the durability | Durability of cognitive and anti-depressive |
| of cognitive and | effects at the Week 10 End of Study (EOS) |
| antidepressant response to | Visit (4 weeks after receiving last dose of |
| Xanamem | trial drug) |
| To determine the treatment | Change from Baseline to EOT in Patient's |
| response of Xanamem | Global Impression of Severity scale (PGI-S) |
| utilizing a patients' global | Change from Baseline to EOT (EOT Visit |
| impression measure | only) in Patient's Global Impression of |
| | Improvement scale (PGI-I) |
| Safety Objectives | Safety Endpoints |
| To evaluate the short-term | Incidence and severity of treatment-emergent |
| | adverse events (TEAEs) |
| safety and tolerability of | Incidence of SAEs and suspected unexpected |
| | serious adverse reactions (SUSARs) |
| Xanamem | Clinically significant changes from Baseline |
| | in: |
| | Clinical safety laboratory evaluations |
| | Vital signs |
| | Physical examinations |
| | Columbia-Suicide Severity Rating Scale |
| | (C-SSRS) |
| Pharmacokinetics (PK) | |
| Objectives | PK Endpoints |
| Confirm Xanamem | Sparse pharmacokinetic sampling for use in |
| exposure and contribute to a | PPK analysis |
| population pharmacokinetic | |
| (PPK) model | |

| Safety Review | A Safety Review Committee (SRC) comprising lead investigator(s), a Sponsor | |
|---|---|---|
| Committee | Medical Monitor, and an Independent Medical Monitor will meet periodically to | |
| | review blinded safety and tolerability data and overall trial conduct. Efficacy will | |
| | not be evaluated or used in any considerations of stopping the trial. The SRC | |
| | Chairperson will be able to request unblinding of individual participants if stopping | |
| | criteria are met, to determine whether the participant(s) who met the stopping | |
| | criteria were randomized to Xanamem. | |
| Inclusion | To be eligible for trial entry, participants must satisfy all of the following criteria: | |
| Criteria: | 1. | Male or female aged 18 to 75, inclusive at the time of Screening. |
| | 2. | Positive MDD primary diagnosis confirmed by MINI at Screening. |
| | 3. | Persistent depressive symptoms as determined by a HAM-D ≥17 at Screening. |
| | 4. | Self-reported subjective cognitive dysfunction, as determined by an affirmative |
| | | response to any aspect of a single-question test about perceived problems with |
| | | their thinking. |
| | 5. | Is either currently or has previously been treated with a stable dose of a first- or |
| | | second-line antidepressant that is approved for the treatment of depression (but |
| | | not a tricyclic antidepressant, monoamine oxidase inhibitor, or vortioxetine). If |
| | | on current treatment, the dose must be stable for at least 6 weeks, and if |
| | | previously treated (self-reported or confirmed by treating physician), the |
| | | treatment must have ceased at least 6 weeks prior to Screening. |
| | 6. | Exhibit a 0.5 SD impairment on the BASIC Boxfiller subtest relative to age and |
| | | education relevant norms at Screening. |
| | 7. | Must provide written informed consent to participate in the trial and be willing |
| | | and able to comply with the requirements of the protocol and complete all trial |
| | | visits. |
| | 8. | Smokers are eligible if they are able to comfortably abstain from nicotine |
| | | (includes vaping) / tobacco products for 2 hours prior to scheduled cognitive |
| | | assessments. |
| | 9. | Participants must be able to comfortably abstain from caffeine intake for |
| | | 4 hours prior to scheduled cognitive assessments. |
| | 10. | Female participants: Unless post-menopausal or male partners are known to be |
| | | infertile, women of childbearing potential (WOCBP) and their partners must |
| | | agree to use an acceptable, highly reliable method of contraception during the |
| | | trial and for 90 days after the last dose of trial treatment. They must have a |
| | | negative serum pregnancy test at Screening and a negative urine pregnancy test |
| | | at Day 1 and must not be breastfeeding, lactating, or planning pregnancy during |
| | | the trial period. WOCBP must agree to use an acceptable form of contraception |
| | | during the treatment period and for at least 90 days after the last dose of trial |
| | | treatment. |
| | 11. | Male participants: Unless known to be infertile, a male participant with a female |
| | | partner of childbearing potential is only eligible to participate if he, and his |
| | | partner, agree to use an acceptable, highly reliable method of contraception |
| | | during the treatment period and for at least 90 days after the last dose of trial |
| | | treatment and if he refrains from donating sperm during this period. |
| Exclusion | | Participants will be excluded from the trial if 1 or more of the following criteria are |
| Criteria: | | applicable: |
| | 1. | Active suicidal ideation within the previous 3 months (‘yes' to either of |
| | | questions 4 or 5 on the C-SSRS or per Investigator assessment). |
| | 2. | On a tricyclic antidepressant, monoamine oxidase inhibitor, esketamine, or |
| | | vortioxetine. |
| | 3. | Other comorbid psychiatric disorder of clinical concern, as determined by |
| | | medical history and Screening assessments. |
| | 4. | A history of clinically diagnosed dementia of any type. |
| | 5. | A history of severe depression treated with electroconvulsive therapy. |
| | 6. | Treatment with transcranial magnetic stimulation within the previous 3 months. |
| | 7. | Has a BMI or body weight that will interfere with participation in the trial, |
| | | including inadequate venous access to complete the trial assessments, to be |
| | | determined at the discretion of the Investigator. |
| | 8. | Abnormalities in resting vital signs at Screening (blood pressure [BP] >160/100 |
| | | mmHg, heart rate [HR] >90 beats per minute [bpm], respiratory rate [RR] >25, |
| | | and clinically significant elevated body temperature), or any clinically |
| | | significant abnormalities in vital signs requiring intervention in the opinion of |
| | | the Investigator. Up to 2 repeat measurements according to Investigator |
| | | discretion are permitted to confirm eligibility. |
| | 9. | The following clinical chemistry values will have an alert flag on the laboratory |

| report and are specifically excluded: | |
|---|---|
| a. | Aspartate aminotransferase >1.5 x the |
| | upper limit of normal (ULN) |
| b. | Alanine aminotransferase >1.5 x ULN |
| c. | Creatinine enzymatic Estimated |
| | Glomerular Filtration Rate |

| | (eGFR) <40 ml/min. |
|---|---|
| 10. | Participants who have a history of liver disease requiring medical intervention, |
| | including fatty liver, or persistent liver function test elevations, unless due to |
| | Gilbert's syndrome. |
| 11. | Previous clinically significant systemic illness or infection, including test |
| | positive COVID-19, within the past 4 weeks prior to Screening, as determined |
| | by the Investigator. |
| 12. | Type I or Type II diabetes requiring insulin. |
| 13. | History of clinically significant hypoglycemia in the past 5 years. |
| 14. | History of disease directly related to the hypothalamus, the pituitary, and/or the |
| | adrenal glands which affects the hypothalamic-pituitary-adrenal (HPA) axis |
| | function, eg, Cushing's Disease, Adenoma, Conn Syndrome, other |
| | malignancies. |
| 15. | Medically diagnosed peripheral neuropathy. |
| 16. | Any medical condition which requires the use of oral corticosteroids >10 |
| | mg/day (prednisolone-equivalent) or high dose inhaled or topical |
| | corticosteroids. |
| 17. | Active malignancy, except for local basal cell carcinoma or squamous cell |
| | carcinoma. |
| 18. | Clinically significant electrocardiogram (ECG) abnormalities, including QTcF |
| | interval >450 msec, (male) and >470 msec (female), other than controlled |
| | atrial fibrillation (on rate control medication with HR ≤90 bpm) at Screening. |
| 19. | Concomitant treatment with stimulant drugs (eg, methylphenidate, |
| | [lis]dexamphetamine) for attention deficit-hyperactivity disorder. |
| 20. | Regular use of benzodiazepines or similar sedative drugs in the daytime. |
| | Nocturnal use, if stable and provided the use is at least 12 hours prior to a trial |
| | visit, is allowed. Use of alprazolam at any time is not permitted. |
| 21. | Chronic opioid therapy with morphine, fentanyl, hydromorphone, tapentadol, |
| | oxycodone, buprenorphine, pethidine, or methadone. Codeine or |
| | immediate-release tramadol is allowed, provided this use is not on days of |
| | cognitive assessments. |
| 22. | Sedative antihistamines (taken in the daytime). |
| 23. | Regular use of St John's wort. |
| 24. | Participation in another clinical trial of a drug or device whereby the last |
| | investigational drug/device administration is within 30 days of Screening, or |
| | 5 half lives of the investigational drug, whichever is longer. |
| 25. | Known allergy to the trial drug (Xanamem) or any of the excipients of the |
| | formulation (lactose, talc, magnesium stearate, croscarmellose sodium). |
| 26. | Trial participants who in the opinion of the Investigator exhibit physical, |
| | cognitive, or language impairments of such severity as to adversely affect the |
| | validity of the data derived from the neuropsychological tests. |
| 27. | Positive testing for HIV, hepatitis B surface antigen, or hepatitis C antibodies at |
| | Screening. Participants returning a positive result will be managed by the site in |
| | line with standard care. |
| 28. | Participants with a history of drug abuse or addiction in the past 2 years, based |
| | on assessment by the Investigator. Positive urinary drug testing results at |
| | Screening not consistent with medical history is an exclusion. |
| 29. | Current use of cannabis/marijuana, or tetrahydrocannabinol-containing |
| | medications. |
| 30. | Evidence or history of alcohol abuse (defined as greater than 21 standard units |
| | per week for males and greater than 14 standard units per week for females). |
| | Participants unwilling to restrict alcohol consumption from Screening until the |
| | end of the trial, to a maximum of 10 standard units per week or 4 standard |
| | drinks per day, are not eligible. |
| 31. | Taking a prohibited medication, primarily high dose corticosteroids, |
| | psychoactive medications, or drugs that may cause peripheral neuropathy. |
| 32. | Any other condition that in the opinion of the Investigator would make it unsafe |

for the participant or difficult to comply with the protocol requirements and trial
procedures.

| | |
|---|---|
| Description of | All participants will receive a single dose of IP (randomized 1:1 to 10 mg Xanamem |
| Trial | or placebo) in the morning with or without food every day for 6 weeks (Day 1 to 42, |
| Intervention: | inclusive). |
| Number of | Approximately 160 participants will be enrolled and randomized into 2 treatment |
| Participants: | groups of approximately equal size. |
| Trial | Participants will take part in the trial for up to 14 weeks. The trial consists of a |
| Duration: | Screening period (maximum 4 weeks), treatment period (6 weeks), and follow-up |
| | period (4 weeks) |
| Statistical | Sample size: |
| Methods: | The sample size for this trial is 160 patients. With allowance for 10% attrition, this |
| | will provide samples of approximately 72 completing participants per treatment |
| | group (10 mg Xanamem, or placebo). |
| | Analysis Populations: |
| | The full analysis set (FAS) population will consist of all randomized participants |
| | who receive at least 1 dose of IP. Efficacy and exploratory efficacy will be examined |
| | in the FAS population. |
| | The per protocol (PP) analysis set is a subset of the FAS population and will consist |
| | of all randomized participants for whom no major protocol deviation or non- |
| | compliance (capsule count or unexpected PK measurements below the lower limit of |
| | quantitation) is documented. The PP population will be used to evaluate the |
| | robustness of the results from the FAS population analyses. |
| | The Safety population includes all participants who are randomized and receive at |
| | least a single dose of trial treatment (active or placebo). |
| | The PK analysis set will consist of all participants in the Safety population who have |
| | at least 1 postdose PK assessment. |
| | Statistical Analysis: |
| | In general, data will be summarized using descriptive statistics (mean, median, SD, |
| | minimum, and maximum) or frequency counts and percentages, as appropriate to the |
| | type of data. |
| | The efficacy analysis for the primary endpoint will assess the difference of |
| | Xanamem from placebo in the change from Baseline in the Attention Composite of a |
| | CTB at Weeks 2, 4, and 6 using a restricted maximum likelihood (REML)-based |
| | mixed model for repeated measures (MMRM) analysis with fixed effects for |
| | treatment, week, and the treatment-by-week interaction, and with baseline score as a |
| | covariate. Pairwise treatment comparisons will be performed at each week; however, |
| | the primary comparison will be the contrast between Xanamem and placebo at Week 6. |
| | MMRM analysis will be used for most other secondary and exploratory endpoints. |
| | Clinical laboratory measurements, adverse events (AEs), vital signs, and physical |
| | examination will be presented via summary descriptive statistics. The C-SSRS and |
| | peripheral neurological examination variables will be presented using summary |
| | statistics according to each variable's scale, and treatment groups will be compared |
| | in an exploratory fashion. |

### Formulations for Xanamem™ and Placebo

TABLE 5
Ingredients of Xanamem ™ and Placebo Capsules, using
representative doses of 0 mg and 10 mg (for Example 2)

| Component | Function | Placebo | 10 mg |
|---|---|---|---|
| Xanamem | Active pharmaceutical | 0 mg | 10 mg |
| | ingredient | | |
| Lactose | Diluent | 180.5 mg | 170.5 mg |
| Croscarmellose | Disintegrant | 7.6 mg | 7.6 mg |
| sodium | | | |
| Talc | Glidant | 0.95 mg | 0.95 mg |
| Magnesium | Lubricant | 0.95 mg | 0.95 mg |
| stearate | | | |

### Questions About Cognition

Potential participants will be asked the following single-question test about perceived cognitive dysfunction to determine eligibility for inclusion in the trial:

‘Since you have been depressed, have you noticed any problems with your thinking?’Thinking problems could include:
poor memory
slowness of thinking
difficulty making decisions
keeping focused
“brain fog”?

Potential participants responding ‘Yes’ to 1 or more of the above symptoms will be eligible for the trial.

### Brief and Simple Index of Cognition Boxfiller Subtest

The BASIC is a brief paper and pencil assessment of the major cognitive domains relevant to neuropsychological research. It is comprised of test paradigms that have previously been shown to be reliable, valid, and sensitive measures of cognition. The Boxfiller subtest is a validated version of the standard symbol matching paradigm where participants are required to match pairs of Japanese characters with Arabic numerals to complete a matrix of boxes. Specifically, it assesses processing speed, associative memory, working memory, attentional skills, and graphomotor speed. The test score reflects the number of digits coded correctly in a 60-second test period.

### Assessments of Cognition

Participants will complete a familiarization session and practice administration for each of the cognitive efficacy assessments (Cogstate CTB tests, HVLT-R, CFT, LFT, and Trail Making A and B) at Screening Visit 1 to ensure understanding of the procedures for the tests and to reduce any potential learning effects during the treatment period (see the Schedule of Assessments [Table 8]).

### Assessment Order

The following order is required for the cognitive and psychiatric assessments, which should be conducted prior to any blood draws:
Cogstate CTB starting with attention composite assessments (Detection, Identification, and One Back tests) and then IDSST-S and One Card Learning
CFT and LFT
HVLT-R
MADRS (if being assessed by a local rater)
Trail Making A and B test
other scales and questionnaires (PSQI, BPI, PGI-S, and PGI-I).

### Assessments

### Cogstate Cognitive Test Battery (CTB)

A customized Cogstate CTB will be used to assess the extent to which 10 mg Xanamem once daily improves performance in multiple cognitive domains (attention, executive function, and episodic memory) from Baseline to each trial visit compared to placebo. The assessments require the participant to perform a variety of digital tasks and take approximately 19 minutes per session, as outlined in Table 7.

TABLE 7
Tests Comprising the Cogstate CTB

| | Approximate Time |
|---|---|
| Test (Cognitive Domain) | Required (minutes) |
| Detection (Psychomotor Function) | 3 |
| Identification (Attention) | 3 |
| One Back (Working Memory) | 4 |
| One Card Learning (Visual Learning) | 6 |
| International Digit Symbol Substitution Test - | 3 |
| Symbols (Processing Speed) | |
| Estimated total time for Cogstate CTB | 19 |

### International Digit Symbol Substitution Test-Symbols (Processing Speed)

The International Digit Symbol Substitution Test is a cognitive test that measures response speed, attention, working memory, and visuospatial processing. The test requires a subject to match symbols to numbers according to a key and copy the symbols into spaces below a row of numbers within a limited time, usually 90 to 180 seconds. The score is the number of correct symbols.

### Hopkins Verbal Learning Test-Revised (HVLT-R)

The HVLT-R is an orally administered list-learning test that will be used to assess the extent to which 10 mg Xanamem improves performance in verbal learning and memory (recognition and recall) from Baseline to each trial visit compared to placebo. The HVLT-R uses a list of 12 nouns with 4 words drawn from each of 3 semantic categories (Benedict 1998), and participants are assessed on their ability to recall the words read aloud by an assessor. The immediate recall component only of the HVLT-R will be used in this trial. The assessment takes approximately 5 to 10 minutes.

### Category Fluency Test (CFT) and Letter Fluency Test (LFT)

The CFT and LFT measure performance in language and executive function domains, and are paper-based verbal fluency tests. The CFT measures spontaneous production of words that are exemplars of different categories (such as animals, fruits, and vegetables) in 1 minute. One (1) category is tested per applicable trial visit, and the same category should not be tested for a given participant at the next visit. The LFT measures spontaneous productions of words beginning with a designated letter (3 different letters are assessed at each applicable trial visit, such as F, A, and S) in 1 minute. The assessment takes approximately 5 minutes. The change in numbers of correct words produced compared to Baseline with 10 mg Xanamem will be compared to placebo.

### Montgomery-Åsberg Depression Rating Scale (MADRS)

The MADRS is a 10-item diagnostic questionnaire used to assess the severity of depressive episodes, and is designed to be sensitive to treatment effects (Montgomery 1979). The MADRS comprises questions on the following symptoms: apparent sadness, reported sadness, inner tension, reduced sleep, reduced appetite, concentration difficulties, lassitude, inability to feel, pessimistic thoughts, and suicidal thoughts. Each item is scored from 0 to 6, and overall scores range from 0 to 60. Higher scores indicate more severe depression. The MADRS will be used to assess the extent to which 10 mg Xanamem improves depressive symptoms from Baseline to each trial visit compared to placebo. It will be administered as an interview to the participant by an accredited health professional and takes 20 to 60 minutes to complete. The MADRS may be administered by a local rater during a trial visit or separately by a central rater during the visit window for relevant trial visits.

### Patient Global Impression of Severity (PGI-S) and Improvement (PGI-I)

The PGI-S and PGI-I enables a participant to provide an assessment of their perception of the severity of their symptoms and any improvements observed during the trial. Participants will be prompted to describe their personal global perception of their depression and cognitive impairment. For the PGI-S, the participant will rate the severity of their symptoms on a seven-point scale: 1, not present; 2, very mild; 3, mild; 4, moderate; 5, moderately severe; 6, severe; 7, extremely severe. The 7-point scale for the PGI-I is rated as 1, very much improved; 2, much improved; 3, minimally improved; 4, no change; 5, minimally worse; 6, much worse; or 7, very much worse. The PGI-S and PGI-I take approximately 5 minutes each to complete.

### Trail Making A and B Test

The Trail Making A and B test is a paper-based test used to assess the extent to which 10 mg Xanamem improves performance in speed of processing and executive function from Baseline to each trial visit compared to placebo. It has demonstrated sensitive results in previous clinical trials in patients with MDD (McIntyre 2014). Part A of the test assesses complex attention and requires a participant to connect randomly positioned numbered circles as quickly as possible. Part B assesses executive function and requires connection of circled letters and numbers in alternating numeric and alphabetic sequence (ie, 1-A-2-B, etc.) as quickly as possible, alternating between numbers and letters. The Trail Making A and B test takes approximately 10 minutes to complete.

### Schedule of Assessments

TABLE 8
Schedule of Assessments

| | Screening | Screening | Baseline | | | EOT | EOS/ET | Unscheduled |
|---|---|---|---|---|---|---|---|---|
| | Visit 1 | Visit 2^{a} | Visit 2 | Visit 3 | Visit 4 | Visit 5 | Visit 6 | Safety Visit^{b} |
| | Week | | | | | | | |
| | −4 to −1 | −1 | 0 | 2 | 4 | 6 | 10 | NA |
| | Day | | | | | | | |
| | −28 to −1 | −13 to −3 | 1 | 14 | 28 | 42 | 70 | NA |
| | Visit Window (Days) | | | | | | | |
| Trial Assessments | NA | NA | NA | ±3 | ±3 | ±5^{c} | ±5 | NA |
| Informed consent | X | | | | | | | |
| Inclusion/exclusion criteria | X | | X | | | | | |
| Medical/medication history, | X | X^{d} | | | | | | |
| demography | | | | | | | | |
| Question about cognition | X | | | | | | | |
| Full physical examination^{e} | X | | | | | | | |
| Abbreviated physical | | | X | X | X | X | X | |
| examination^{e} | | | | | | | | |
| Peripheral neurological | X | | (X) | (X) | (X) | (X) | (X) | |
| examination^{e} | | | | | | | | |
| Height | X | | | | | | | |
| Weight | X | | X | | | X | X | |
| Vital signs^{f} | X | | X | X | X | X | X | X |
| 12-lead electrocardiogram^{g} | X | | (X) | (X) | (X) | (X) | (X) | |
| Clinical safety labs^{h} | X | X | X | X | X | X | X | |
| Viral serology | X | | | | | | | |
| Pregnancy test/FSH testⁱ | X | X | X | X | X | X | X | |
| C-SSRS | X | | X | X | X | X | X | X |
| Drugs of abuse (urine) | X | | | | | | | |
| Mini-International | X | | | | | | | |
| Neuropsychiatric Interview ^{j} | | | | | | | | |
| BASIC Boxfiller subtest | X | | | | | | | |
| Hamilton Depression Rating | X | | | | | | | |
| Scale | | | | | | | | |
| Randomization | | | X | | | | | |
| Cogstate Cognitive Test | X | | X^{m} | X | X | X | X | |
| Battery^{k, l} | | | | | | | | |
| CFT and LFT^{k, l} | X | | X | X | X | X | X | |
| Hopkins Verbal Learning | X | | X | X | X | X | X | |
| Test - Revised^{k, l} | | | | | | | | |
| Montgomery-Åsberg | | | X | X | X | X | X | |
| Depression Rating Scale^{k, l} | | | | | | | | |
| PGI-S^{k, n} | | | X | X | X | X | X | |
| PGI-I^{k, n} | | | | | | X | | |
| Trail Making A and B tests^{k, l} | X | | X | | | X | | |
| IP dispensing | | | X | | | | | |
| IP administration at site^{o} | | | X | | | | | |
| IP administration | | | X | X | X | X | | |
| IP compliance and | | | | X | X | X | | |
| accountability^{p} | | | | | | | | |
| Adverse event reporting | | | X | X | X | X | X | X |
| Concomitant medications | X | | X | X | X | X | X | X |
| PK blood sample^{q} | | | | X | X | X | | |

Abbreviations: β-HCG = beta-human chorionic gonadotrophin; BASIC = brief and simple index of cognition; C-SSRS = Columbia-Suicide Severity Rating Scale; ECG = electrocardiogram; EOS = End of Study; EOT = End of Treatment Visit; ET = Early Termination; FSH = follicle-stimulating hormone; IP = investigational product; NA = not applicable; PGI-I = Patient Global Impression of Improvement scale; PGI-S = Patient Global Impression of Severity scale; PK = pharmacokinetics; (X) = only if clinically indicated.
^{a}Participants whose Screening Visit occurred >14 days prior to their scheduled Day 1 will be required to attend the trial site between Day −13 to Day −3 for a Screening Visit 2 to collect additional Screening blood samples and for review of medical history.
^{b}Any other additional unspecified assessments may be conducted at an Unscheduled Safety Visit at the discretion of the Investigator.
^{c}The EOT visit should be scheduled to take place on or after the final day of treatment (Day 42 [+5 days]) to ensure that the full course of IP is completed, taking visit windows into account.
^{d}Medical history only to be reviewed at the Screening Visit 2.
^{e}The peripheral neurological examination will be performed at Screening as part of the full physical examination, and at other visits only if clinically indicated.
^{f}Up to 2 repeat measurements according to Investigator discretion are permitted at Screening to confirm eligibility. At all other times, an additional repeat measurement should be performed and used as the reported measurement if the initial BP and/or HR are elevated.
^{g}ECGs will be performed at Screening. ECGs at subsequent treatment visits (Baseline, Week 2, Week 4, Week 6, and Follow-up Visit) should only be performed if clinically indicated.
^{h}Clinical safety labs include hematology and clinical chemistry. Where possible, clinical safety labs should be taken last after other participant-facing activities.
^{I}Women of childbearing potential will be screened for pregnancy. The Screening pregnancy test(s) will be by blood sample to detect the presence of β-HCG (collected as part of clinical chemistry sample). At all other visits, urine dipstick will be used to detect pregnancy. FSH will be measured in post-menopausal women at Screening to confirm menopausal status.
^{j} The Mini-International Neuropsychiatric Interview (MINI) will be administered by an accredited health professional during the Screening period to determine eligibility. This assessment should be performed after other general medical assessments undertaken during Screening.
^{k}Multiple efficacy tests should be conducted in the same order at each visit for each participant, with the order documented in source documents.
^{l}A familiarization session and practice administration for each cognitive efficacy assessment will be performed at Screening for familiarization/learning purposes.
^{m}The Cogstate CTB assessment will be conducted twice during the Baseline Visit 2 (Day 1), at least one hour apart.
ⁿPGI-S will be performed at multiple visits, whereas the PGI-I will only be conducted at EOT (Visit 6).
^{o}On Day 1, participants will take their first dose of IP (Xanamem or placebo) at the site with or without food. Food may be provided either by the trial site or the participant. Each other day during the treatment period, participants should take their IP at home in the morning with or without food, including on days of trial follow-up visits.
^{p}Participants will be required to bring their bottles of placebo/IP with them to the EOT Visit for calculation of trial drug accountability, and their completed drug intake diary with them to all visits during the treatment period for calculation of IP compliance.
^{q}Samples for PK analysis can be taken at any convenient time during trial visits.
Note:
For consistency of assessments, all visits should be scheduled in the morning (am) if possible.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of treating or preventing depression in a subject resistant to antidepressant treatment, comprising administering to the subject a therapeutically effective amount of a compound of Formula I, or a pharmaceutically acceptable salt, solvate or prodrug thereof: 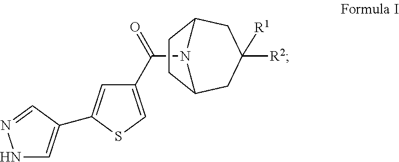
wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR₃, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are each independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR₅, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

2. A method of treating or preventing depression associated cognitive impairment in a subject resistant to antidepressant treatment, comprising administering to the subject a therapeutically effective amount of a compound of Formula I, or a pharmaceutically acceptable salt, solvate or prodrug thereof: 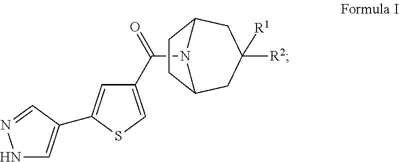
wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR₃, —NR³R⁴, —COR³, —CO₂R³, —CONR³R⁴, —NR³COR⁴, —SO₂R³, —SO₂NR³R⁴, and —NR³SO₂R⁴; wherein R³ and R⁴ are each independently selected from the group consisting of hydrogen, C₁₋₆alkyl, 3-7-membered carbocyclyl and 3-7-membered heterocyclyl; wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR₅, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

3. The method of claim 1 and claim 2, wherein the treatment is for depression and depression associated cognitive impairment in the subject.

4. The method of any one of claims 1 to 3, wherein the compound of Formula I is a compound of Formula Ia, or a pharmaceutically acceptable salt, solvate or prodrug thereof: 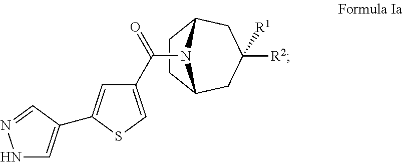
wherein R¹ and R² are each independently selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, 3-10-membered carbocyclyl, 3-10-membered heterocyclyl, —CN, —CF₃, —OR³, —SR₃, —NR³R⁴, —COR³, —CO₂R³, —CONR³R⁴, —NR³COR⁴, —SO₂R³, —SO₂NR³R⁴, and —NR³SO₂R⁴;
wherein R³ and R⁴ are independently selected from the group consisting of hydrogen, C₁₋₆alkyl, 3-7-membered carbocyclyl and 3-7-membered heterocyclyl;
wherein each 3-10-membered carbocyclyl, 3-10-membered heterocyclyl, 3-7-membered carbocyclyl, and 3-7-membered heterocyclyl, is unsubstituted or substituted with one or more substituents selected from the group consisting of hydrogen, halogen, C₁₋₆alkyl, —O—C₁₋₆alkyl, C₁₋₆haloalkyl, —O—C₁₋₆haloalkyl, C₂₋₆alkenyl, C₂₋₆alkynyl, —CN, —CF₃, —OR⁵, —SR₅, —NR⁵R⁶, —COR⁵, —CO₂R⁵, —CONR⁵R⁶, —NR⁵COR⁶, —SO₂R⁵, —SO₂NR⁵R⁶, and —NR⁵SO₂R⁶; and wherein each R⁵ and R⁶ are independently selected from the group consisting of hydrogen and C₁₋₆alkyl.

5. The method of any one of claims 1 to 4, wherein R¹ and R² are each independently selected from the group consisting of hydrogen, halogen, 3-10-membered carbocyclyl, 3-10-membered heterocyclyl, —OH, —CN, and —NH₂; and wherein if present, each 3-10-membered carbocyclyl and 3-10-membered heterocyclyl may be further substituted with one or more substituents selected from the group consisting of hydrogen, halogen, —OH, —CN, —CF₃, —NH₂, and C₁₋₆alkyl.

6. The method of any one of claims 1 to 5, wherein R¹ and R² are each independently selected from the group consisting of hydrogen, halogen, 6-membered carbocyclyl, 6-membered heterocyclyl, —OH, —CN, and —NH₂; and wherein if present, each 6-membered carbocyclyl and 6-membered heterocyclyl may be further substituted with one or more substituents selected from the group consisting of hydrogen, halogen, —OH, —CN, —CF₃, —NH₂, and C₁₋₆alkyl.

7. The method of any one of claims 1 to 6, wherein R¹ is selected from the group consisting of hydrogen, halogen, —OH, —CN, —CF₃, —NH₂, and C₁₋₆alkyl, and R² is independently selected from the group consisting of:

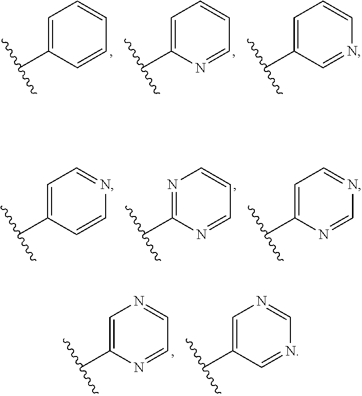

8. The method of any one of claims 1 to 7, wherein the compound of Formula I is selected from the group consisting of:

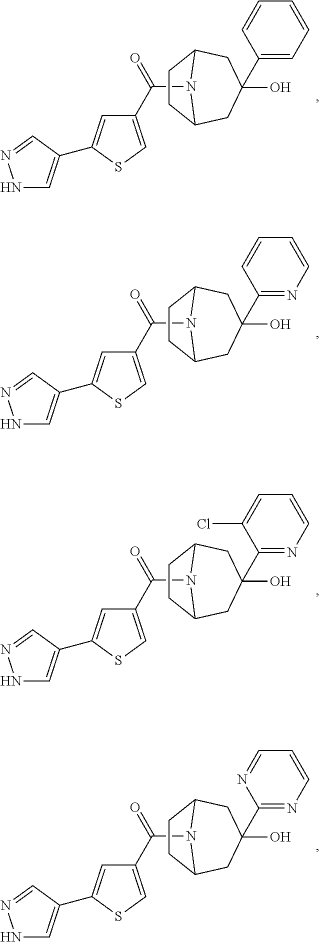 [SMILES: O=C(c1csc(-c2cn[nH]c2)c1)N1C2CCC1CC(O)(c1ccccc1)C2 ; O=C(c1csc(-c2cn[nH]c2)c1)N1C2CCC1CC(O)(c1ccccn1)C2 ; O=C(c1csc(-c2cn[nH]c2)c1)N1C2CCC1CC(O)(c1ncccc1Cl)C2 ; O=C(c1csc(-c2cn[nH]c2)c1)N1C2CCC1CC(O)(c1ncccn1)C2]

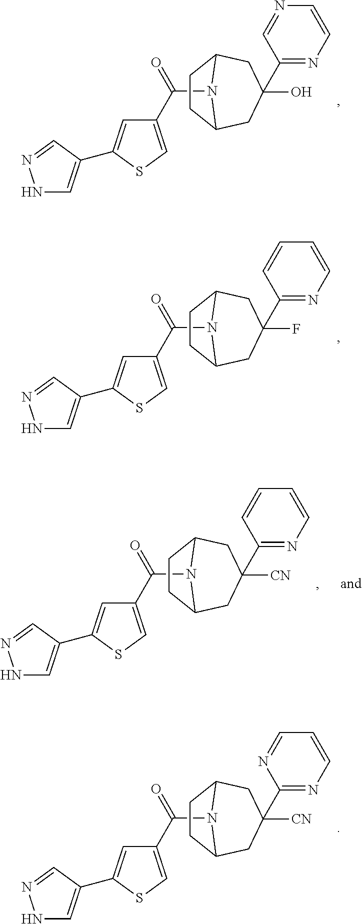 [SMILES: O=C(c1csc(-c2cn[nH]c2)c1)N1C2CCC1CC(O)(c1cnccn1)C2 ; O=C(c1csc(-c2cn[nH]c2)c1)N1C2CCC1CC(F)(c1ccccn1)C2 ; N#CC1(c2ccccn2)CC2CCC(C1)N2C(=O)c1csc(-c2cn[nH]c2)c1 ; N#CC1(c2ncccn2)CC2CCC(C1)N2C(=O)c1csc(-c2cn[nH]c2)c1]

9. The method of any one of claims 1 to 8, wherein the compound of Formula I is:

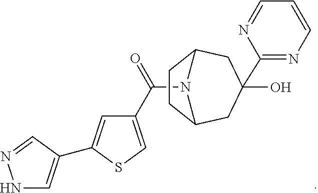 [SMILES: O=C(c1csc(-c2cn[nH]c2)c1)N1C2CCC1CC(O)(c1ncccn1)C2]

10. The method of any one of claims 1 to 9, wherein the compound of Formula Ia is:

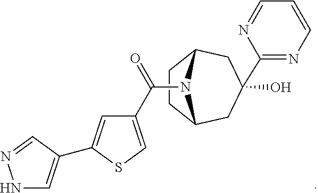 [SMILES: O=C(c1csc(-c2cn[nH]c2)c1)N1[C@@H]2CC[C@H]1C[C@](O)(c1ncccn1)C2]

11. The method of any one of claims 1 to 10, wherein the subject resistant to antidepressant treatment is resistant to one or more antidepressants selected from the group consisting of serotonin specific reuptake inhibitors (SSRI), noradrenaline reuptake inhibitors (NARI), noradrenaline and specific serotonergic antidepressants (NaSSA), melatonergic agonists (MA), serotonin and noradrenaline reuptake inhibitors (SNRI), reversible inhibitors of monoamine oxidase (RIMAs), tricyclic antidepressants (TCA), mono-amine oxidase inhibitors (MAOI), and serotonin modulators (SM).

12. The method of any one of claims 1 to 11, wherein the subject resistant to antidepressant treatment is resistant to one or more antidepressants selected from the group consisting of imipramine, amitriptyline, dothiepin, desipramine, nortriptyline, doxepin, protriptyline, trimipramine, maprotiline, amoxapine, trazodone, bupropion, chlomipramine, fluoxetine, citalopram, sertraline, paroxetine, tianeptine, nefazadone, venlafaxine, desvenlafaxine, duloxetine, reboxetine, mirtazapine, phenelzine, tranylcypromine, moclobemide, reboxetine, mianserin, agomelatine, and vortioxetine.

13. The method of any one of claims 1 to 12, wherein the subject resistant to antidepressant treatment is resistant to one or more antidepressants selected from the group consisting of selective serotonin reuptake inhibitors (SSRI).

14. The method of claim 13, wherein the SSRI is selected from citalopram, escitalopram, fluoxetine, fluvoxamine, paroxetine, sertraline, dapoxetine, indalpine, zimelidine, alaproclate, centpropazine, cericlamine, femoxetine, ifoxetine, omiloxetine, panuramine, pirandamine, and seproxetine.

15. The method of claim 13, wherein the SSRI is selected from citalopram, escitalopram, fluoxetine, fluvoxamine, paroxetine, sertraline.

16. The method of any one of claims 1 to 15, wherein the subject resistant to antidepressant treatment is resistant to one or more antidepressants selected from the group consisting of noradrenaline reuptake inhibitors (NARI).

17. The method of claim 16, wherein the NARI is reboxetine.

18. The method of any one of claims 1 to 17, wherein the subject resistant to antidepressant treatment is resistant to one or more antidepressants selected from the group consisting of noradrenaline and specific serotonergic antidepressants (NaSSA).

19. The method of claim 18, wherein the NaSSA is selected from mirtazapine and mianserin.

20. The method of any one of claims 1 to 19, wherein the subject resistant to antidepressant treatment is resistant to one or more antidepressants selected from the group consisting of melatonergic agonists (MA).

21. The method of claim 20, wherein the MA is agomelatine.

22. The method of any one of claims 1 to 21, wherein the subject resistant to antidepressant treatment is resistant to one or more antidepressants selected from the group consisting of serotonin and noradrenaline reuptake inhibitors (SNRI).

23. The method of claim 22, wherein the SNRI is selected from atomoxetine, desvenlafaxine, duloxetine, levomilnacipran, milnacipran, sibutramine, tramadol, and venlafaxine.

24. The method of any one of claims 1 to 23, wherein the subject resistant to antidepressant treatment is resistant to one or more antidepressants selected from the group consisting of reversible inhibitors of monoamine oxidase (RIMAs).

25. The method of claim 24, wherein the RIMA is moclobemide.

26. The method of any one of claims 1 to 25, wherein the subject resistant to antidepressant treatment is resistant to one or more antidepressants selected from the group consisting of tricyclic antidepressants (TCA).

27. The method of claim 26, wherein the TCA is selected from amitriptyline, clomipramine, dothiepin, imipramine, nortriptyline, and doxepin.

28. The method of any one of claims 1 to 27, wherein the subject resistant to antidepressant treatment is resistant to one or more antidepressants selected from the group consisting of mono-amine oxidase inhibitors (MAOI).

29. The method of claim 28, wherein the MAOIs is selected from phenelzine and tranylcypromine.

30. The method of any one of claims 1 to 29, wherein the subject resistant to antidepressant treatment is resistant to one or more antidepressants selected from the group consisting of serotonin modulators (SM).

31. The method of claim 30, wherein the SM is vortioxetine.

32. The method of any one of claims 1 to 31, wherein the subject resistant to antidepressant treatment is a subject resistant to treatment of major depressive disorder (MDD).

33. The method of claim 32, wherein the MDD is diagnosed by identifying a subject having a HAM-D of at least 17 or a DSM-V criteria using Mini International Neuropyschiatric Interview of at least 20.

34. The method of any one of claims 1 to 33, wherein the administration of a compound of Formula I, or a pharmaceutically acceptable salt, solvate or prodrug thereof, provides an improvement of depression in the treated subject relative to a non-treated subject.

35. The method of any one of claims 1 to 34, wherein an improvement of depression in the treated subject is determined using MADRS testing criteria.

36. The method of any one of claims 1 to 35, wherein the depression associated cognitive impairment in a subject resistant to antidepressant treatment is a subject having at least 0.5 SD impairment on WAIS Coding Test relative to age, education and gender.

37. The method of any one of claims 1 to 36, wherein the administration of a compound of Formula I, or a pharmaceutically acceptable salt, solvate or prodrug thereof, provides improved cognitive ability of the treated subject relative to a non-treated subject using one or more testing criteria methods selected from Cogstate Neuropyschological Testing criteria, Hopkins Verbal Learning Test, Controlled Word Association Test, and Trail Making A and B Tests.

38. The method of claim 37, wherein the administration of a compound of Formula I, or a pharmaceutically acceptable salt, solvate or prodrug thereof, provides an improvement by showing a change in baseline in Executive Function and/or Episodic Memory.

39. The method of any one of claims 1 to 37, wherein the compound of Formula I, or a pharmaceutically acceptable salt, solvate or prodrug thereof, is administered in amount so as to deliver a total daily dosage of from about 1 to 70 mg of Formula I or from about 5 to 40 mg of Formula I.

40. The method of any one of claims 1 to 39, wherein the compound of Formula I, or a pharmaceutically acceptable salt, solvate or prodrug thereof, is administered as a single once-daily dosage or a twice-daily dosage.

41. The method of any one of claims 1 to 40, wherein the compound of Formula I, or a pharmaceutically acceptable salt, solvate or prodrug thereof, is administered as a twice-daily dosage of 10 mg per dosage.

42. The method of any one of claims 1 to 41, wherein the compound of Formula I, or a pharmaceutically acceptable salt, solvate or prodrug thereof, is administered orally or is administered orally with food.

43. The method of any one of claims 1 to 42, wherein the subject is between 18 to 75 years of age.
